(19) United States
(12) Patent Application Publication
(10) Pub. No.: US 2022/0299510 A1
(43) Pub. Date: Sep. 22, 2022

(54) METHODS FOR DIAGNOSING, PROGNOSING AND MONITORING TREATMENT FOR THROMBOSIS IN SUBJECTS WITH SYSTEMIC LUPUS ERYTHEMATOSUS

(71) Applicant: Exagen Inc., Vista, CA (US)

(72) Inventor: Thierry Dervieux, Vista, CA (US)

(21) Appl. No.: 17/633,152

(22) PCT Filed: Aug. 12, 2020
(86) PCT No.: PCT/US2020/045982

§ 371 Date: Feb. 4, 2022

Related U.S. Application Data
(60) Provisional application No. 62/885,612, filed on Aug. 12, 2019.
(60) Provisional application No. 63/002,055, filed on Mar. 30, 2020.

(51) Int. Cl. G01N 33/564; G01N 33/543

(52) U.S. Cl. CPC G01N 33/564; G01N 33/54306; G01N 2800/104; G01N 2800/226; G01N 2800/50; G01N 2800/52

(57) ABSTRACT

Provided herein are methods for the diagnosis, prognosis, and treatment of thrombosis in subject having or suspected of having systemic lupus erythematosus including determination of a level of platelet-bound complement C4d, C3 level, and one or both of a level of antiphosphatidyl serine/prothrombin complex and/or lupus anticoagulant.

### CROSS-REFERENCED APPLICATIONS

This application claims priority benefit to U.S. Provisional Application No. 62/885,612 filed Aug. 12, 2019 and U.S. Provisional Application No. 63/002,055 filed Mar. 30, 2020, which are incorporated in their entireties by reference herein.

### BACKGROUND OF THE INVENTION

The excessive risk of thrombosis in SLE is dependent on the presence of abnormalities that are specific for the disease, including low C3, antiphospholipid (aPL antibodies (in particular lupus anticoagulant [LAC]) and nephrotic syndrome. Other factors related to SLE treatment such as prednisone may further elevate the risk of thrombosis.

Cell-bound complement activation products (CB-CAPs) and deposition of C4d split fragments on haematopoietic cells such as B lymphocytes (BC4d) and erythrocyte (EC4d) are commonly present in SLE. In contrast, deposition of C4d on platelets (PC4d) is generally uncommon (20% SLE) but highly specific. (See, for example, Refs. 1-2).

There remains the need to identify thrombosis risk in SLE patients.

### BRIEF SUMMARY OF THE INVENTION

In an aspect, provided herein are methods for diagnosing thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibodies in a biological sample from the subject; and/or (ii) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the subject has risk of thrombosis.

In an aspect, provided herein are methods for prognosing development of thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibodies in a biological sample from the subject; and/or (ii) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the subject is at risk of developing thrombosis.

In an aspect, provided herein are methods for monitoring treatment for thrombosis in a subject having Systemic Lupus Erythematosus (SLE) and being treated for thrombosis, including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibodies in a biological sample from the subject; and/or (ii) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the treatment for thrombosis has not been effective. The combination of (i) a level of PC4d in the biological sample at or below a threshold PC4d level, (ii) a level of complement C3 at or above a threshold C3 level, and (iii) a level of anti-PS/PT IgG antibody at or below a threshold anti-PS/PT IgG antibody level and/or a level of LAC antibody in the biological sample above a threshold LAC antibody level, indicates that the treatment for thrombosis has been effective.

In an aspect, provided herein are methods of detecting a marker in a Systemic Lupus Erythematosus (SLE) subject that has or is suspected of having thrombosis, the method including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibody in a biological sample from the subject; and/or (ii) a level of lupus anticoagulant (LAC) in a biological sample from the subject.

In an aspect, provided herein are methods of treating thrombosis in a Systemic Lupus Erythematosus (SLE) subject including determining: (a) a level of PC4d in a first blood sample from the subject; (b) a level of complement C3 protein in a second blood sample from the subject; and (c) one or both of: (i) a level of anti-phosphatidyl serine/prothrombin (PS/PT) IgG antibody in a third blood sample from the subject; and/or (ii) a level of lupus anticoagulant (LAC) in a fourth blood sample from the subject, wherein the first, second, third and fourth blood samples may be the same or different; and (d) treating the subject with an effective amount of one or more antithrombotic therapeutic selected from hydroxychloroquine, heparin, dalteparin, fondaparinux, enoxaparin, warfarin, dabigatran, rivaroxaban, apixiban, betrixaban and edoxaban.

In an aspect, provided herein are methods for preparing a sample from a Systemic Lupus Erythematosus (SLE) subject useful for analyzing a plurality of markers involved in thrombosis, including: (a) collecting whole blood from the subject; (b) producing a platelet fraction derived from the whole blood comprising lysing red blood cells, and measuring a level of PC4d in the platelet fraction; and (c) producing a first serum or plasma fraction from the whole blood and measuring a level of C3 in said serum or plasma fraction; and (d) producing a second serum or plasma fraction from the whole blood and measuring a level of PS/PT complex antibody in said second serum or plasma fraction.

### BRIEF DESCRIPTION OF THE DRAWINGS

FIGS. 1A-B show percent patients with any thrombosis, venous thrombosis and arterial thrombosis. The composite score (range 0-3) corresponds to the number of abnormalities present at the time of specimen collection. FIG. 1A shows abnormal PC4d (>20 net MFI), low C3 (<81 mg/dl) and LAC (dRVVT >37 seconds) (n=143). FIG. 1B shows abnormal PC4d (>20 net MFI), low C3 (<81 mg/dl) and anti-PS/PT IgG (>30 units) (n=148).

FIGS. 2A-E present the composite score of risk factors and thrombosis. Percent patients with any thrombosis, venous thrombosis and arterial thrombosis is given. Panel A shows abnormal PC4d (>20 net MFI) and LAC (dRVVT >37 seconds) (n=143); Panel B shows abnormal PC4d (>20 net MFI) and low C3 (<81 mg/dl) (n=148); Panel C shows abnormal PC4d (>20 net MFI) and anti-PS/PT IgG (>30 units) (n=149); Panel D shows low C3 (<81 mg/dl) and LAC (dRVVT >37 seconds) (n=143); Panel E shows Low C3 (<81 mg/dl) and anti-PS/PT IgG (>30 units) (n=148).

FIGS. 3A-B presents data demonstrating the relationships between persistency in PC4d, risk score and thrombosis during follow-up (FU). Panel A shows results from logistic regression analysis, which revealed that the percentage FU visits with abnormal PC4d status significantly associated with any thrombosis (OR range 11.7 CI 95%: 3.23-42.44) (p<0.001), venous thrombosis (OR range=33.4 CI95: 5.8-193.5) (p<0.001) and approached significance with arterial thrombosis (OR=4.7 CI 95%: 0.9-25.4) (p=0.08). Panel B shows results during FU, the mean risk score per patient was 0.57±0.76 (n=149). Risk score at FU associated with any thrombosis (OR=3.8 CI 95%: 2.0-7.2 per unit change, OR range=53.9 CI 95%: 7.8-372.4) (p<0.001), venous thrombosis (OR=3.9 CI 95%: 1.9-8.2 per unit change, OR range=60.3 CI95%: 6.5-560.8) and arterial thrombosis (OR=3.0 CI95%: 1.4-6.4 per unit change, OR range=26.5 CI95%: 2.7-259.4).

### DETAILED DESCRIPTION OF THE INVENTION

### Definitions

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein might be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose. The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise.

Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment described herein. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2, or 1% to a reference quantity, level, value, concentration, measurement, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of ” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

As used herein, the terms “disease” or “condition” are used in accordance with their plain and ordinary meaning and refer to a state of being or health status of a patient or subject capable of being treated with the compounds or methods provided herein. The disease may be an autoimmune disease. In some instances, the disease is systemic lupus erythematosus. The disease may be an inflammatory disease. The disease may be a cardiovascular disease. In some instances, the condition is thrombosis.

As used herein, the term “Systemic Lupus Erythematosus” or “SLE” are used in accordance with its plain and ordinary meaning and refer to an autoimmune disease, characterized by the production of unusual autoantibodies in the blood. These autoantibodies bind to their respective antigens, forming immune complexes which circulate and eventually deposit in tissues. This immune complex deposition causes chronic inflammation and tissue damage.

As used herein, the term “thrombosis” is used in accordance with its plain and ordinary meaning and refers to the formation of a blood clot inside a blood vessel, obstructing the flow of blood through the circulatory system. When a blood vessel (a vein or an artery) is injured, the body uses platelets (thrombocytes) and fibrin to form a blood clot to prevent blood loss. Even when a blood vessel is not injured, blood clots may form in the body under certain conditions. A clot, or a piece of the clot, that breaks free and begins to travel around the body is known as an embolus. Thrombosis may occur in veins (venous thrombosis) or in arteries (arterial thrombosis). Venous thrombosis leads to congestion of the affected part of the body, while arterial thrombosis (and rarely severe venous thrombosis) affects the blood supply and leads to damage of the tissue supplied by that artery (ischemia and necrosis).

As used herein, the term “diagnosis” is used in accordance with its plain and ordinary meaning and refers to an identification or likelihood of the presence of thrombosis or outcome in a subject.

As used herein, the term “prognosis” is used in accordance with its plain and ordinary meaning and refers to the likelihood or risk of a subject developing a particular outcome or particular event, such as thrombosis.

As used herein, a “biological sample” is used in accordance with its plain and ordinary meaning and encompasses essentially any sample type that can be used in a diagnostic or prognostic method described herein. The biological sample may be any bodily fluid, tissue or any other sample obtained from a subject or subject's body from which clinically relevant protein marker levels or antibody levels may be determined. The definition encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polypeptides or proteins. The term “biological sample” encompasses a clinical sample, but also, in some instances, includes cells in culture, cell supernatants, cell lysates, blood, serum, plasma, urine, cerebral spinal fluid, biological fluid, and tissue samples. The sample may be pretreated as necessary by dilution in an appropriate buffer solution or concentrated, if desired. In embodiments, the biological sample is a blood sample. In embodiments, the biological sample is whole blood, plasma, or serum. In embodiments, the biological sample is whole blood. In embodiments, the biological sample is plasma. In embodiments, the biological sample is serum.

As used herein, the term “whole blood” is used in accordance with its plain and ordinary meaning and refers to blood drawn directly from the body from which none of the components, such as plasma or platelets, has been removed.

As used herein, the terms “plasma” and “blood plasma” are used in accordance with their plain and ordinary meaning and refers to the yellowish liquid component of blood that holds the blood cells in whole blood in suspension. It is the liquid part of the blood that carries cells and proteins throughout the body. It makes up about 55% of the body's total blood volume. Blood plasma is separated from the blood by spinning a tube of fresh blood containing an anticoagulant in a centrifuge until the blood cells fall to the bottom of the tube. The blood plasma is then poured or drawn off.

As used herein, the term “serum” is used in accordance with its plain and ordinary meaning and refers to the fluid and solute component of blood which does not play a role in clotting. It may be defined as blood plasma without fibrinogens. Serum includes all proteins not used in blood clotting; all electrolytes, antibodies, antigens, hormones; and any exogenous substances (e.g., drugs or microorganisms). Serum does not contain white blood cells (leukocytes), red blood cells (erythrocytes), platelets, or clotting factors.

As used herein, the terms “treating” or “treatment” (and as well understood in the art) are used in accordance with their plain and ordinary meaning and broadly includes any approach for obtaining beneficial or desired results in a subject's condition, including clinical results. Beneficial or desired clinical results can include, but are not limited to, alleviation or amelioration of one or more symptoms or conditions, diminishment of the extent of a disease, stabilizing (i.e., not worsening) the state of disease, prevention of a disease's transmission or spread, delay or slowing of disease progression, amelioration or palliation of the disease state, diminishment of the reoccurrence of disease, and remission, whether partial or total and whether detectable or undetectable. In other words, “treatment” as used herein includes any cure, amelioration, or prevention of a disease. Treatment may prevent the disease from occurring; inhibit the disease's spread; relieve the disease's symptoms, fully or partially remove the disease's underlying cause, shorten a disease's duration, or do a combination of these things.

As used herein, the terms “treating” and “treatment” may include prophylactic treatment. Treatment methods include administering to a subject a therapeutically effective amount of an active agent. The administering step may consist of a single administration or may include a series of administrations. The length of the treatment period depends on a variety of factors, such as the severity of the risk or condition, the age of the patient, the concentration of active agent, the activity of the compositions used in the treatment, or a combination thereof. It will also be appreciated that the effective dosage of an agent used for the treatment or prophylaxis may increase or decrease over the course of a particular treatment or prophylaxis regime. Changes in dosage may result and become apparent by standard diagnostic assays known in the art. In some instances, chronic administration may be required. For example, the compositions are administered to the subject in an amount and for a duration sufficient to treat the patient.

As used herein, the term “prevent” is used in accordance with its plain and ordinary meaning and refers to a decrease in the occurrence of disease symptoms in a patient. The prevention may be complete (no detectable symptoms) or partial, such that fewer symptoms are observed than would likely occur absent treatment.

As used herein, the terms “patient” or “subject in need thereof” or “subject” are used in accordance with their plain and ordinary meaning and refer to a living organism suffering from or prone to a disease or condition that can be treated by administration of a pharmaceutical composition. Non-limiting examples include humans, other mammals, bovines, rats, mice, dogs, monkeys, goat, sheep, cows, deer, and other non-mammalian animals. In some embodiments, a subject is human.

As used herein, the term “control” or “control experiment” are used in accordance with their plain and ordinary meaning and refer to an experiment in which the subjects or reagents of the experiment are treated as in a parallel experiment except for omission of a procedure, reagent, or variable of the experiment. In some instances, the control is used as a standard of comparison in evaluating experimental effects. In some embodiments, a control is the measurement of the activity of a protein in the absence of a compound as described herein (including embodiments and examples). In some instances, the control is a quantification standard used as a reference for assay measurements. The quantification standard may be a synthetic protein, a recombinantly expressed purified protein, a purified protein isolated from its natural environment, a protein fragment, a synthesized polypeptide, or the like.

As described herein, the terms “marker”, “protein marker”, “polypeptide marker,” and “biomarker” are used interchangeably throughout the disclosure, and are used in accordance with their plain and ordinary meaning. As used herein, a protein marker refers generally to a protein or polypeptide, the level or concentration of which is associated with a particular biological state, particularly a state associated with a cardiovascular disease, event or outcome. Panels, assays, kits and methods described herein may comprise antibodies, binding fragments thereof or other types of target-binding agents, which are specific for the protein marker described herein.

As used herein, the terms “polypeptide” and “protein”, may be used interchangeably, are used in accordance with their plain and ordinary meaning, and refer to a polymeric form of amino acids of any length, which can include coded and non-coded amino acids, chemically or biochemically modified or derivatized amino acids, and polypeptides having modified peptide backbones. In various embodiments, detecting the levels of naturally occurring protein marker proteins in a biological sample is contemplated for use within diagnostic, prognostic, or monitoring methods disclosed herein. The term also includes fusion proteins, including, but not limited to, naturally occurring fusion proteins with a heterologous amino acid sequence, fusions with heterologous and homologous leader sequences, with or without N-terminal methionine residues; immunologically tagged proteins; and the like. The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

As used herein, the term “antibody” is used in accordance with its plain and ordinary meaning and in the broadest sense. The term specifically covers, but is not limited to, monoclonal antibodies, polyclonal antibodies, multi-specific antibodies (e.g., bispecific antibodies) formed from at least two intact antibodies, single chain antibodies (e.g., scFv), and antibody fragments or other derivatives, so long as they exhibit the desired biological specificity. The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

As used herein, the term “monoclonal antibody” is used in accordance with its plain and ordinary meaning and refers to an antibody obtained from a population of substantially homogeneous antibodies, i.e., the individual antibodies comprising the population are identical except for possible naturally occurring mutations that can be present in minor amounts. In certain specific embodiments, the monoclonal antibody is an antibody specific for a protein marker described herein.

As used herein, the term “detectably labeled antibody” is used in accordance with its plain and ordinary meaning and refers to an antibody (or antibody fragment) which retains binding specificity for a protein marker described herein, and which has an attached detectable label. The detectable label can be attached by any suitable means, e.g., by chemical conjugation or genetic engineering techniques. Methods for production of detectably labeled proteins are well known in the art. Detectable labels may be selected from a variety of such labels known in the art, including, but not limited to, haptens, radioisotopes, fluorophores, paramagnetic labels, enzymes (e.g., horseradish peroxidase), or other moieties or compounds which either emit a detectable signal (e.g., radioactivity, fluorescence, color) or emit a detectable signal after exposure of the label to its substrate. Various detectable label/substrate pairs (e.g., horseradish peroxidase/diaminobenzidine, avidin/streptavidin, and luciferase/luciferin)), methods for labeling antibodies, and methods for using labeled antibodies are known in the art.

As used herein, the term “specifically (or selectively) binds” to an antibody or “specifically (or selectively) immunoreactive with,” when referring to a protein or peptide, is used in accordance with its plain and ordinary meaning and refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, immunoassays are routinely used to select antibodies specifically immunoreactive with a protein.

An example immunoglobulin (antibody) structural unit comprises a tetramer. Each tetramer is composed of two identical pairs of polypeptide chains, each pair having one “light” (about 25 kDa) and one “heavy” chain (about 50-70 kDa). The N-terminus of each chain defines a variable region of about 100 to 110 or more amino acids primarily responsible for antigen recognition. The terms “variable heavy chain,” “VH,” or “VH” refer to the variable region of an immunoglobulin heavy chain, including an Fv, scFv, dsFv or Fab, while the terms “variable light chain,” “VL” or “VL” refer to the variable region of an immunoglobulin light chain, including of an Fv, scFv, dsFv or Fab.

As used herein, the term “functional fragments” is used in accordance with its plain and ordinary meaning and in the context of antibodies, refers to those fragments that retain sufficient binding affinity and specificity for a protein marker to permit a determination of the level of the protein marker in a biological sample. In some cases, a functional fragment will bind to a protein marker with substantially the same affinity and/or specificity as an intact full chain molecule from which it may have been derived. Examples of antibody functional fragments include, but are not limited to, complete antibody molecules, antibody fragments, such as Fv, single chain Fv (scFv), complementarity determining regions (CDRs), VL (light chain variable region), VH (heavy chain variable region), Fab, F(ab)2′ and any combination of those or any other functional portion of an immunoglobulin peptide capable of binding to target antigen. As appreciated by one of skill in the art, various antibody fragments can be obtained by a variety of methods, for example, digestion of an intact antibody with an enzyme, such as pepsin, or de novo synthesis. Antibody fragments are often synthesized de novo either chemically or by using recombinant DNA methodology. Thus, the term antibody, as used herein, includes antibody fragments either produced by the modification of whole antibodies, or those synthesized de novo using recombinant DNA methodologies (e.g., single chain Fv) or those identified using phage display libraries.

For specific proteins described herein, the named protein includes any of the protein's naturally occurring forms, variants or homologs that maintain the protein transcription factor activity (e.g., within at least 50%, 80%, 90%, 95%, 96%, 97%, 98%, 99% or 100% activity compared to the native protein). In some embodiments, variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring form.

As used herein, the term “complement system” also known as “complement cascade” is used in accordance with its plain and ordinary meaning and refers to a part of the immune system that enhances (complements) the ability of antibodies and phagocytic cells to clear microbes and damaged cells from an organism, promote inflammation, and attack the pathogen's cell membrane. The complement system consists of a number of small proteins that are synthesized by the liver, and circulate in the blood as inactive precursors. When stimulated by one of several triggers, proteases in the system cleave specific proteins to release cytokines and initiate an amplifying cascade of further cleavages. The end result of this complement activation or complement fixation cascade is stimulation of phagocytes to clear foreign and damaged material, inflammation to attract additional phagocytes, and activation of the cell-killing membrane attack complex. Over 30 proteins and protein fragments make up the complement system, including serum proteins, and cell membrane receptors.

As used herein, the term “classical pathway” or “classical complement pathway” are used in accordance with their plain and ordinary meaning and refer to one of three biochemical pathways activate the complement system. The classical pathway is triggered by activation of the Cl-complex. The Cl-complex is composed of 1 molecule of Clq, 2 molecules of Clr and 2 molecules of Cls, or C1qr²s². This occurs when Clq binds to IgM or IgG complexed with antigens. A single pentameric IgM can initiate the pathway, while several, ideally six, IgGs are needed. This also occurs when Clq binds directly to the surface of the pathogen. Such binding leads to conformational changes in the Clq molecule, which leads to the activation of two Clr molecules. Clr is a serine protease. They then cleave Cls (another serine protease). The Clr²s² component now splits C4 and then C2, producing C4a, C4b, C2a, and C2b (historically, the larger fragment of C2 was called C2a but is now referred to as C2b). C4b and C2a bind to form the classical pathway C3-convertase (C4b2a complex), which promotes cleavage of C3 into C3a and C3b. C3b later joins with C4b2a to make C5 convertase (C4b2a3b complex).

As used herein, the term “cell-bound complement activation products” or “CB-CAPS” refers to hydrolyzed complement activation products bound to circulating cells such as erythrocytes, platelets, B and T lymphocytes.

As used herein, the term “platelet-bound C4d” or “PC4d” refers to C4d products of complement activation deposited on platelets.

As used herein, the terms “Complement component 3” and “C3” are used in accordance with their plain and ordinary meaning and refer to a protein of the immune system of the same name. C3 plays a central role in the activation of the complement system and contributes to innate immunity Its activation is required for both classical and alternative complement activation pathways.

As used herein, the terms “lupus anticoagulant” and “LAC” are used in accordance with their plain and ordinary meaning and refer to an immunoglobulin that binds to phospholipids and proteins associated with the cell membrane. Lupus anticoagulant in living systems can cause an increase in inappropriate blood clotting

As used herein, the terms “anti-phosphatidylserine/prothrombin complex” and “anti-PS/PT” are used in accordance with their plain and ordinary meaning and refer to an antiphospholipid antibody. In embodiments, antibodies to phosphatidylserine/prothrombin complex include IgG and IgM.

As used herein, the terms “enzyme-linked immunosorbent assay” and “ELISA” are used in accordance with their plain and ordinary meaning and refer to a commonly used analytical biochemistry assay. The assay uses a solid-phase enzyme immunoassay (EIA) to detect the presence of a ligand (commonly a protein) in a liquid sample using antibodies directed against the protein to be measured. ELISA has been used as a diagnostic tool in medicine, plant pathology, and biotechnology, as well as a quality control check in various industries. In the most simple form of an ELISA, antigens from the sample are attached to a surface. Then, a matching antibody is applied over the surface so it can bind to the antigen. This antibody is linked to an enzyme, and in the final step, a substance containing the enzyme's substrate is added. The subsequent reaction produces a detectable signal, most commonly a color change.

As used herein, the term “immunoturbidimetry” is used in accordance with its plain and ordinary meaning and refers to a technique which relies upon the light scattering characteristics of antigen/antibody complexes. For example, when a patient specimen, containing an antigen (such as C3c or C4), is combined with antiserum, a complex between the antigen and the antiserum will form. When light is directed through this suspension, a portion of the light will be transmitted and focused onto an optic device (photodiode via an optical lens system). The amount of transmitted light observed by the optic device is indirectly proportional to the protein concentration (C3c or C4) in the patient specimen. Therefore, a specimen that contains a high concentration of C3c or C4 would transmit less light than a specimen that contains a low concentration of C3c or C4.

As used herein, the term “score” refers to a numerical values assigned to a result as it relates diagnostic or prognostic determinations. A score can be a positive, intermediate, or negative diagnostic score. A score can be a positive, intermediate, or negative prognostic score. One or multiple cutoffs can be used with the score to determine specific levels of risk. In embodiments, a score is algorithmically derived based on normalized and/or mathematically transformed values, such as protein concentrations, the presence/absence of clinical factors, vital statistics, or ratios of different factors. The algorithm which generates the score can be ratio-based, cut-off-based, linear or non-linear, including decision tree or rule-based models. In embodiments, a thrombotic risk score assigns 1 point for every abnormality, so it can be 0, 1, 2, or 3. It may not be less than 0. Cumulatively, the presence of PC4d, low C3 and LAC abnormalities as a composite risk score was higher in the presence of thrombosis (1.93±0.25) than in its absence (0.81±0.06) (p<0.01); a score of 0 can be considered negative in terms of risk of thrombosis.

As further described herein, the “training set” is the set of patients or patient samples that are used in the process of training (i.e., developing, evaluating and building) the final diagnostic or prognostic model. The “validation set” is a set of patients or patient samples that are withheld from the training process, and are only used to validate the performance of the final diagnostic or prognostic model. If the set of patients or patient samples are limited in number, all available data may be used as a training set, or as an “in-sample” validation set.

As used herein, the term “normalized” refers to a type of transformation where the values are designed to fit a specific distribution, typically so that they are similar to the distributions of other variables. For example, for hypothetical proteins A and B, the raw concentration of protein A ranges from 0 to 500 and the raw concentration of Protein B ranges from 0 to 20,000, it is not trivial looking at thee raw values to determine which one is “higher”. For instance, is 400 of Protein A higher than 15,000 of Protein B. By conducting a normalization process, the concentrations are rescaled so that they are on the same scale: centered at zero, with a variance of 1. Thus, it becomes a routine exercise to determine which one is higher because the normalized concentrations are comparable. Many learning algorithms work better on data that are normalized; otherwise, in this example for instance, Protein B might get more weight in the algorithm because it has higher values even if it were not empirically “higher.”

As used herein, the term “transformed” refers to a mathematical process applied to a numerical value, regardless of the input or output value. It may include taking protein concentrations and calculating the base-10 logarithm from original values, reflecting a “log-transformation.”

The phrase “one or both of: (i) a level of anti-phosphatidyl serine/prothrombin (PS/PT) IgG antibody in a biological sample from the subject; and (ii) a level of lupus anticoagulant (LAC) in a biological sample from the subject” means any one of the following: (1) a level of anti-phosphatidyl serine/prothrombin (PS/PT) IgG antibody in a biological sample from the subject; (2) a level of lupus anticoagulant (LAC) in a biological sample from the subject; or (3) a level of anti-phosphatidyl serine/prothrombin (PS/PT) IgG antibody in a biological sample from the subject and a level of lupus anticoagulant (LAC) in a biological sample from the subject.

The phrase “and/or” means either or both. For example, X and/or Y means either (1) X; or (2) Y; or (3) X and Y.

### I. Methods

In an aspect, provided herein are methods for diagnosing thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibodies in a biological sample from the subject; and/or (ii) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the subject has risk of thrombosis.

In an aspect, provided herein are methods for prognosing development of thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibodies in a biological sample from the subject; and/or (ii) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the subject is at risk of developing thrombosis.

In an aspect, provided herein are methods for monitoring treatment for thrombosis in a subject having Systemic Lupus Erythematosus (SLE) and being treated for thrombosis, including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibody in a biological sample from the subject; and/or (ii) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the treatment for thrombosis has not been effective. The combination of (i) a level of PC4d in the biological sample at or below a threshold PC4d level, (ii) a level of complement C3 at or above a threshold C3 level, and (iii) a level of anti-PS/PT IgG antibody at or below a threshold anti-PS/PT IgG antibody level and/or a level of LAC antibody in the biological sample above a threshold LAC antibody level, indicates that the treatment for thrombosis has been effective.

In an aspect, provided herein are methods of detecting a marker in a Systemic Lupus Erythematosus (SLE) subject that has or is suspected of having thrombosis, the method including determining: (a) a level of platelet-bound C4d (PC4d) protein in a biological sample from the subject; (b) a level of complement C3 protein in a biological sample from the subject; and (c) one or both of: (i) a level of anti-phosphatidyl serine/prothrombin (PS/PT) IgG antibody in a biological sample from the subject; and/or (ii) a level of lupus anticoagulant (LAC) in a biological sample from the subject.

In embodiments, the methods provided herein include a subject. In embodiments, the subject is a human. In embodiments, the subject has Systemic Lupus Erythematosus (SLE). In embodiments, the subject is suspected of having Systemic Lupus Erythematosus (SLE). In embodiments, the subject has Systemic Lupus Erythematosus (SLE) and has had a thrombosis. In embodiments, the subject has Systemic Lupus Erythematosus (SLE) and is at risk of having or developing thrombosis.

Any suitable biological sample from the subject may be used. In embodiments, the biological sample is a blood, whole blood, serum, or plasma sample from a subject. In embodiments, the biological sample is a blood sample from a subject. In embodiments, the biological sample is a whole blood sample from a subject. In embodiments, the biological sample is a serum sample from a subject. In embodiments, the biological sample is a plasma sample from a subject.

In some embodiments, a blood sample is treated with EDTA (ethylene diamine-tetraacetate) to inhibit complement activation. In embodiments, samples are maintained at room temperature. In embodiments, samples are stored at 4° C. The collected blood may separated into components methods known in the art. For example, centrifugation may be used to separate whole blood into plasma and red cells or under a lower centrifugal force, to separate it into plasma, buffy coat (used to make platelets), and red blood cells.

In embodiments, a whole blood sample may be fractionated into different components. In embodiments, a whole blood sample is centrifuged to isolate plasma. In embodiments, the whole blood is treated with a coagulant, centrifuged to remove clots and blood cells, and the resulting liquid supernatant is serum.

In embodiments, red blood cells are separated from other cell types in the sample by differential centrifugation. In embodiments, the whole blood is treated with a lysing agent to lyse red blood cells and obtain a platelet fraction. Platelet isolation can be performed with methods known in the art, including differential centrifugation or immunoprecipitation using antibodies specific for platelets (e.g., CD42b).

The level (e.g., quantity or amount) of a particular biomarker or protein marker or component can be measured in a sample using a variety of methods known to those of skill in the art. Such methods include, but are not limited to, flow cytometry, ELISA, and the like. In one embodiment, the determination of the level of PC4d and C3 is made using flow cytometric methods, with measurements taken by direct or indirect immunofluorescence using polyclonal or monoclonal antibodies specific for each of the molecules. Each of these molecules can be measured with a separate sample (e.g., platelet-specific fractions) or using a single sample (e.g., whole blood).

In embodiments, determining a level includes processing a biological sample from a subject and detecting levels or amounts of a particular component of the sample. In embodiments, determining a level includes processing a biological sample and detecting a levels of PC4d in the sample.

In embodiments, the biological sample is a platelet fraction of whole blood. In embodiments, the platelet fraction can be processed for analysis of platelet-bound complement activation products, such as PC4d. In embodiments, the platelet-bound complement activation products are measured by fluorescence-activated cell sorting (FACS). In embodiments, the platelet-bound complement activation product is PC4d.

In embodiments, PC4d levels are measured by FACS as follows: red blood cells from a whole blood sample treated with EDTA are lysed and platelets are stained using mouse monoclonal antibody against human C4d, or alternatively using mouse IgG1 kappa monoclonal (MOPC-21), isotype control. After incubation for 30 min at 2° C.-8° C., samples are stained using goat antimouse conjugated to Fluorescein Isothiocyanate (FITC) (30 min at 2° C.-8° C. in the dark). A monoclonal antibody against human CD42b conjugated to phycoerythrin (PE) (platelet-specific marker) may be used to identify C4d complement activation fragment covalently bound to the platelets. PACS analysis may be performed using a Gallios (10-colors) flow cytometer (Beckman Coulter, Brea, Calif.) equipped with a CAP software to measure fluorescent staining intensity. Light scatter (forward and side) gating parameters were used during acquisition to isolate the platelet population, followed by secondary gating based on positive CD42b PE staining. Quantification of the non-specific (isotype control) and specific (C4d) fluorescence in the FL1 (fluorescein isothiocyanate) channel may be used to determine the CD42b PE gated platelet cells (5000 events). Net mean fluorescence intensity (MFI) may be determined by subtraction of isotype control background MFI results from the specific C4d MFI results on gated platelet cells.

In embodiments, determining a level includes processing a biological sample from a subject and detecting a level of complement C3 in the sample. Low complement C3 status may be established using any suitable method, including but not limited to immunoturbidity, chemiluminescence, and the like. In embodiments, the biological sample is serum and C3 levels are measured using immunoturbidimetry.

In embodiments, immunoturbidimetry includes in vitro measurement of serum C3c and C4. In embodiments, immunoturbidimetry includes in vitro measurement of serum C3. In embodiments, immunoturbidity assays are performed on a device or instrument, for example the Optilite Clinical Chemistry Analyzer. In embodiments, measurements of C3 concentrations are reported in milligrams per deciliter (mg/dL), and are calculated by reference to a calibration curve that is maintained within the instrument operating software.

In embodiments, determining a level includes processing a biological sample from a subject and determining a level of anti-phosphatidyl serine/prothrombin (PS/PT) IgG antibody and/or IgM antibody. In embodiments, the biological sample is whole blood. In embodiments, the biological sample is serum. In embodiments, the biological sample is plasma. Anti-PS/PT complex antibodies may be measured using any suitable means, including but not limited to immunoassays. In embodiments, a level of anti-phosphatidyl serine/prothrombin (PS/PT) IgG and/or IgM antibody is measured by enzyme-linked immunosorbent assay. In embodiments, enzyme-linked immunosorbent assays (ELISA) for the detection of IgG and IgM class antibodies to phosphatidylserine/prothrombin complex (PS/PT) in serum or plasma may be semi-quantitative and qualitative.

In embodiments, microwell plate wells are coated with purified PS/PT complex and then stabilized. Upon incubation, serum containing PS/PT IgG or PS/PT IgM antibodies bind with the PS/PT. Unbound protein is removed by washing and anti-human IgG or IgM horseradish peroxidase (HRP) labeled conjugate is added to the wells. After incubation, the unbound conjugate is removed by washing. A peroxidase substrate is then added, which undergoes a color change in the presence of the conjugated enzyme. After stopping the enzymatic production of colored product, the presence or absence of prothrombin antibody is determined spectrophometrically by measuring and comparing the color intensity that develops in the patient wells with that of a five point calibration curve. Units as defined by the device or instrument (for example Inova ELISA: QUANTA Lite™ aPS/PT IgG and/or QUANTA Lite™ aPS/PT IgM kits).

In embodiments, determining a level includes processing a biological sample from a subject and determining a level of lupus anticoagulant (LAC). In embodiments, determining a level of LAC includes performing a coagulation based assay. In embodiments, a coagulation-based assay include dilute Russell viper venom test (dRVVT) and an activated partial thromboplastin time (aPTT).

In embodiments, provided herein are methods of diagnosing thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) a level of anti-phosphatidyl serine/prothrombin (PS/PT) IgG antibodies in a biological sample from the subject. In embodiments, the combination of (i) a level of PC4d in the biological sample above a threshold PC4d level, (ii) a level of complement C3 below a threshold C3 level, and (iii) a level of anti-PS/PT IgG antibody above a threshold anti-PS/PT IgG antibody level indicates that the subject has risk of thrombosis.

In embodiments, provided herein are methods of diagnosing thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of LAC in the biological sample above a threshold LAC level, indicates that the subject has risk of thrombosis.

In embodiments, provided herein are methods for prognosing development of thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) a level of anti-phosphatidyl serine/prothrombin (PS/PT) IgG antibodies in a biological sample from the subject. The combination of (i) a level of PC4d in the biological sample above a threshold PC4d level, (ii) a level of complement C3 below a threshold C3 level, and (iii) a level of anti-PS/PT IgG antibody above a threshold anti-PS/PT IgG antibody level, indicates that the subject is at risk of developing thrombosis.

In embodiments, provided herein are methods for prognosing development of thrombosis in a subject having Systemic Lupus Erythematosus (SLE), including determining: (a) a level of platelet-bound C4d (PC4d) protein in a biological sample from the subject; (b) a level of complement C3 protein in a biological sample from the subject; and (c) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of LAC in the biological sample above a threshold LAC level, indicates that the subject is at risk of developing thrombosis.

In embodiments, provided herein are methods for monitoring treatment for thrombosis in a subject having Systemic Lupus Erythematosus (SLE) and being treated for thrombosis, including determining: (a) a level of platelet-bound C4d (PC4d) protein in a biological sample from the subject; (b) a level of complement C3 protein in a biological sample from the subject; and (c) a level of anti-phosphatidyl serine/prothrombin (PS/PT) IgG antibody in a biological sample from the subject. In embodiments, a combination of (i) a level of PC4d in the biological sample above a threshold PC4d level, (ii) a level of complement C3 below a threshold C3 level, and (iii) a level of anti-PS/PT IgG antibody above a threshold anti-PS/PT IgG antibody level, indicates that the treatment for thrombosis has not been effective. In embodiments, a combination of (i) a level of PC4d in the biological sample at or below a threshold PC4d level, (ii) a level of complement C3 at or above a threshold C3 level, and (iii) a level of anti-PS/PT IgG antibody at or below a threshold anti-PS/PT IgG antibody level, indicates that the treatment for thrombosis has been effective.

In embodiments, provided herein are methods for monitoring treatment for thrombosis in a subject having Systemic Lupus Erythematosus (SLE) and being treated for thrombosis, including determining: (a) a level of platelet-bound C4d (PC4d) protein in a biological sample from the subject; (b) a level of complement C3 protein in a biological sample from the subject; and (c) a level of lupus anticoagulant (LAC) in a biological sample from the subject. The combination of (i) a level of PC4d in the biological sample above a threshold PC4d level, (ii) a level of complement C3 below a threshold C3 level, and (iii) a level of LAC in the biological sample above a threshold LAC level, indicates that the treatment for thrombosis has not been effective. The combination of (i) a level of PC4d in the biological sample at or below a threshold PC4d level, (ii) a level of complement C3 at or above a threshold C3 level, and (iii) a level of LAC antibody in the biological sample above a threshold LAC antibody level, indicates that the treatment for thrombosis has been effective.

In embodiments, the methods include determining a level of PC4d protein in a whole blood biological sample from a subject. In embodiments, the methods include determining the level of PC4d protein in a platelet fraction derived from a whole blood biological sample from a subject. In embodiments, determining the level of PC4d is determined using an antibody specific for C4d in flow cytometry. In the various embodiments described herein, a threshold PC4d level is ≥20 mean fluorescence intensity (WI) units measured using flow cytometry.

In embodiments, the methods include determining a level of complement C3 protein in a serum sample from a subject. In embodiments, a level of C3 is determined using an antibody specific for C3 in an immunoturbidity assay. In the various embodiments described herein, a threshold C3 marker level is <81 mg protein per deciliter (mg/dl) serum measured using a C3-specific antibody.

In embodiments, the methods include determining a level of anti-PS/PT antibody is in a serum, plasma, or whole blood sample. In embodiments, a level of anti-PS/PT IgG is determined using an enzyme-linked immunosorbent assay (ELISA). In embodiments, a level of anti-PS/PT IgM is determined using an enzyme-linked immunosorbent assay (ELISA). In the various embodiments described herein, a threshold anti-PS/PT IgG level is >30 Units measured using ELISA.

In embodiments, the methods include determining a level of lupus anticoagulant. In embodiments, a level of LAC is determined using a coagulation assay. In the various embodiments described herein, a threshold of LAC is determined using the dilute Russell's viper venom time (dRVVT >37 s). In the various embodiments described herein, a threshold of LAC is determined using a cutoff of 37 seconds to establish positivity of the dRVVT test. In other words, if sample does not clot within 37 seconds, it is considered positive. In some embodiments, a threshold of LAC is determined using ratio between the time that it takes the patient sample to clot divided by the time it takes for a normal sample to clot, and a ratio >1.3 is considered positive.

In embodiments, the methods provided herein include where the PC4d level, C3 level, and one or both of the anti-PS/PT IgG antibody and LAC levels are determined 2, 3, 4, or more times. In embodiments, the methods provided herein include where the PC4d level, C3 level, and one or both of the anti-PS/PT IgG antibody and LAC levels are determined twice. In embodiments, the methods provided herein include where the PC4d level, C3 level, and one or both of the anti-PS/PT IgG antibody and LAC levels are determined 3 times. In embodiments, the methods provided herein include where the PC4d level, C3 level, and one or both of the anti-PS/PT IgG antibody and LAC levels are determined 4 times. In embodiments, the methods provided herein include where the PC4d level, C3 level, and one or both of the anti-PS/PT IgG antibody and LAC levels are determined more than 4 times.

In embodiments, the methods provided herein include where the PC4d level, C3 level, and anti-PS/PT IgG antibody level is determined 2, 3, 4 or more times. In embodiments, the methods provided herein include where the PC4d level, C3 level, and anti-PS/PT IgG antibody level is determined twice. In embodiments, the methods provided herein include where the PC4d level, C3 level, and anti-PS/PT IgG antibody level is determined 3 times. In embodiments, the methods provided herein include where the PC4d level, C3 level, and anti-PS/PT IgG antibody level is determined 4 times. In embodiments, the methods provided herein include where the PC4d level, C3 level, and anti-PS/PT IgG antibody level is determined more than 4 times

In embodiments, the methods provided herein include where the PC4d level, C3 level, and LAC level is determined 2, 3, 4 or more times. In embodiments, the methods provided herein include where the PC4d level, C3 level, and LAC level is determined twice. In embodiments, the methods provided herein include where the PC4d level, C3 level, and LAC level is determined 3 times. In embodiments, the methods provided herein include where the PC4d level, C3 level, and LAC level is determined 4 times. In embodiments, the methods provided herein include where the PC4d level, C3 level, and LAC level is determined 4 or more times.

In embodiments, the methods provided herein include where the subject is being treated with hydroxychloroquine (HCQ), and where the method further includes determining a level of HCQ in a whole blood sample from the subject. In embodiments, an HCQ level below a threshold HCQ whole blood level indicates that the subject, is at risk of venous thrombosis, and/or indicates efficacy of HCQ and any other anti-thrombotic therapy. In embodiments, the threshold HCQ whole blood level is 500 ng/ml.

In embodiments, the various methods described herein further include communication of the diagnosis, prognosis, or indication of treatment effect via a remote patient monitoring (RPM) internet-based devices to a medical professional, including but not limited to the subject's doctor and/or the subject's pharmacy for automated ordering of an anti-thrombotic therapeutic, including but not limited to an oral anti-coagulant.

In embodiments, the methods provided herein include where the subject is identified as having thrombosis, at risk of thrombosis, or in need of modified therapy for thrombosis, where the method further includes treating the subject with an anti-thrombotic therapeutic, or increasing the dosage of an anti-thrombotic therapeutic. In embodiments, an anti-thrombotic therapeutic is selected from hydroxychloroquine, heparin, dalteparin, fondaparinux, enoxaparin, warfarin, dabigatran, rivaroxaban, apixiban, betrixaban and edoxaban. In embodiments, an anti-thrombotic therapeutic is hydroxychloroquine. In embodiments, an anti-thrombotic therapeutic is heparin. In embodiments, an anti-thrombotic therapeutic is dalteparin. In embodiments, an anti-thrombotic therapeutic is fondaparinux. In embodiments, an anti-thrombotic therapeutic is enoxaparin. In embodiments, an anti-thrombotic therapeutic is warfarin. In embodiments, an anti-thrombotic therapeutic is dabigatran. In embodiments, an anti-thrombotic therapeutic is rivaroxaban. In embodiments, an anti-thrombotic therapeutic is apixiban. In embodiments, an anti-thrombotic therapeutic is betrixaban. In embodiments, an anti-thrombotic therapeutic is edoxaban.

In an aspect, provided herein are methods of treating thrombosis in a Systemic Lupus Erythematosus (SLE) subject including determining: (a) a level of PC4d in a first blood sample from the subject; (b) a level of complement C3 protein in a second blood sample from the subject; and (c) one or both of: (i) a level of anti-phosphatidyl serine/prothrombin (PS/PT) IgG antibody in a third blood sample from the subject; and/or (ii) a level of lupus anticoagulant (LAC) in a fourth blood sample from the subject, wherein the first, second, third and fourth blood samples may be the same or different; and (d) treating the subject with an effective amount of one or more antithrombotic therapeutic selected from hydroxychloroquine, heparin, dalteparin, fondaparinux, enoxaparin, warfarin, dabigatran, rivaroxaban, apixiban, and edoxaban.

In embodiments, the biological samples in the different determining steps may be the same biological sample or different biological samples. In embodiments, the biological samples are different fractions of a biological sample derived from a single subject. In embodiments, the biological samples in the different determining steps are different samples and are referred to as a first biological sample, second biological sample, etc.

In embodiments, methods of treating thrombosis in an SLE subject include determining: (a) a level of PC4d in a first blood sample from the subject; (b) a level of complement C3 protein in a second blood sample from the subject; and (c) a level of anti-phosphatidyl serine/prothrombin (PS/PT) IgG antibody in a third blood sample from the subject, where the first, second, and third blood samples may be the same or different; and (d) treating the subject with an effective amount of one or more antithrombotic therapeutic.

In embodiments, determining a level of PC4d in a first blood sample from the subject includes any one of the various methods described herein. In embodiments, determining a level of a level of complement C3 protein in a second blood sample from the subject includes any of the various methods described herein. In embodiments, determining a level of anti-phosphatidyl serine/prothrombin (PS/PT) IgG antibody includes any of the various methods described herein. In embodiments, determining a level of PC4d above a threshold of ≥20 mean fluorescence intensity (MFI) units measured using flow cytometry; determining a level of C3 below a threshold of <81 mg protein per deciliter (mg/dl) serum measured using a C3-specific antibody, and determining a level of anti-PS/PT IgG above a threshold of >30 Units measured using ELISA provides a determination of risk of thrombosis in the subject.

In embodiments, methods of treating thrombosis in an SLE subject include determining: (a) a level of PC4d in a first blood sample from the subject; (b) a level of complement C3 protein in a second blood sample from the subject; and (c) a level of LAC in a third blood sample from the subject, where the first, second, and third blood samples may be the same or different; and (d) treating the subject with an effective amount of one or more antithrombotic therapeutic. In embodiments, determining a level of anti-phosphatidyl serine/prothrombin (PS/PT) IgG antibody includes any of the various methods described herein. In embodiments, determining a level of PC4d above a threshold of ≥20 mean fluorescence intensity (MFI) units measured using flow cytometry; determining a level of C3 below a threshold of <81 mg protein per deciliter (mg/dl) serum measured using a C3-specific antibody, and determining a level of LAC using a coagulation assay provides a determination of risk of thrombosis in the subject.

In embodiments, the methods for treating thrombosis in an SLE subject determined to have a risk of thrombosis includes administering an anti-thrombotic therapeutic selected from hydroxychloroquine, heparin, dalteparin, fondaparinux, enoxaparin, warfarin, dabigatran, rivaroxaban, apixiban, betrixaban and edoxaban. In embodiments, an anti-thrombotic therapeutic is hydroxychloroquine. In embodiments, an anti-thrombotic therapeutic is heparin. In embodiments, an anti-thrombotic therapeutic is dalteparin. In embodiments, an anti-thrombotic therapeutic is fondaparinux. In embodiments, an anti-thrombotic therapeutic is enoxaparin. In embodiments, an anti-thrombotic therapeutic is warfarin. In embodiments, an anti-thrombotic therapeutic is dabigatran. In embodiments, an anti-thrombotic therapeutic is rivaroxaban. In embodiments, an anti-thrombotic therapeutic is apixiban. In embodiments, an anti-thrombotic therapeutic is betrixaban. In embodiments, an anti-thrombotic therapeutic is edoxaban.

In an aspect, provided herein are methods for preparing a sample from a Systemic Lupus Erythematosus (SLE) subject useful for analyzing a plurality of markers involved in thrombosis, including: (a) collecting whole blood from the subject; (b) producing a platelet fraction derived from the whole blood comprising lysing red blood cells, and measuring a level of PC4d in the platelet fraction; and (c) producing a first serum or plasma fraction from the whole blood and measuring a level of C3 in the first serum or plasma fraction; and (d) producing a second serum or plasma fraction from the whole blood and measuring a level of anti-PS/PT complex antibody in the second serum or plasma fraction.

In embodiments, the methods for preparing a sample include collecting whole blood from a subject and producing a platelet fraction. Producing a platelet fraction may be accomplished by any method known in the art. In embodiments, producing a platelet fraction includes lying red blood cells and using a platelet specific antibody. In embodiments, measuring a level of PC4d includes any of the various embodiments described herein. In embodiments, measuring a level of PC4d includes using a platelet specific antibody and a C4d antibody includes fluorescence activated cell sorting.

In embodiments, the methods for preparing a sample include producing a first serum or plasma fraction from the whole blood of a subject and measuring a level of C3 in the first serum or plasma fraction. In embodiments, measuring a level of C3 includes any of the various embodiments described herein. In embodiments, measuring the level of C3 includes an immunoturbidity assay.

In embodiments, the methods for preparing a sample include producing a first serum or plasma fraction from the whole blood of a subject and measuring a level of anti-PS/PT complex antibody. In embodiments, measuring a level of anti-PS/PT complex antibody includes any of the various embodiments described herein. In embodiments, measuring a level of anti-PS/PT complex antibody an immunoassay.

### P-1 Embodiments

Embodiment P-1. A method for diagnosing thrombosis in a subject having or at risk of Systemic Lupus Erythematosus (SLE), comprising determining a marker level combination of:

(a) platelet C4d (PC4d) in a biological sample from the subject;

(b) complement C3 in a biological sample from the subject; and

(c) one or both of:

(i) anti-phosphatidyl serine/prothrombin (PS/PT) IgG antibodies in a biological sample from the subject; and/or

(ii) lupus anticoagulant (LAC) in a biological sample from the subject;

wherein a marker level combination of (i) a level of PC4d in the biological sample above a threshold PC4d level, (ii) a level of complement C3 below a threshold C3 level, and (iii) a level of one or both of anti-PS/PT IgG antibodies above a threshold anti-PS/PT IgG antibody level and/or a level of LAC in the biological sample above a threshold LAC level, indicates that the subject has risk thrombosis.

Embodiment P-2. A method for prognosing development of thrombosis in a subject having or at risk of Systemic Lupus Erythematosus (SLE), comprising determining a marker level combination of:

(a) platelet C4d (PC4d) in a biological sample from the subject;

(b) complement C3 in a biological sample from the subject; and

(c) one or both of:

(i) anti-phosphatidyl serine/prothrombin (PS/PT) IgG antibodies in a biological sample from the subject; and/or

(ii) lupus anticoagulant (LAC) in a biological sample from the subject;

wherein a marker level combination of (i) a level of PC4d in the biological sample above a threshold PC4d level, (ii) a level of complement C3 below a threshold C3 level, and (iii) a level of one or both of anti-PS/PT IgG antibodies above a threshold anti-/PT IgG antibody level and/or a level of LAC in the biological sample above a threshold LAC level, indicates that the subject is at risk of thrombosis.

Embodiment P-3. A method for monitoring treatment for thrombosis in a subject having Systemic Lupus Erythematosus (SLE) and being treated for thrombosis, comprising determining a marker level combination of:

(a) platelet C4d (PC4d) in a biological sample from the subject;

(b) complement C3 in a biological sample from the subject; and

(c) one or both of:

(i) anti-phosphatidyl serine/prothrombin (PS/PT) IgG antibodies in a biological sample from the subject; and/or

(ii) lupus anticoagulant (LAC) in a biological sample from the subject;

(I) wherein a marker level combination of (i) a level of PC4d in the biological sample above a threshold PC4d level, (ii) a level of complement C3 below a threshold C3 level, and (iii) a level of anti-PS/PT antibodies above a threshold anti-PS/PT IgG antibody level and/or a level of LAC in the biological sample above a threshold LAC level, indicates that the treatment for thrombosis has not been effective, and/or

(II) wherein marker level combination of (i) a level of PC4d in the biological sample at or below a threshold PC4d level, (ii) a level of complement C3 at or above a threshold C3 level, and (iii) a level of anti-PS/PT IgG antibodies at or below a threshold anti-PS/PT IgG antibody level and/or a level of LAC in the biological sample above a threshold LAC level, indicates that the treatment for thrombosis has been effective.

Embodiment P-4. The method of any one of Embodiments P-1-P-3, wherein the marker level combination comprises a level of anti-PS/PT IgG antibodies from a biological sample.

Embodiment P-5. The method of any one of Embodiments P-1-P-4, wherein the level of PC4d is measured in a serum or whole blood biological sample.

Embodiment P-6. The method of any one of Embodiments P-1-P-5, wherein the level of complement C3 in measured a serum sample.

Embodiment P-7. The method of any one of Embodiments P-1-P-6, wherein the level of anti-PS/PT antibodies is measured in a serum, plasma, or whole blood sample.

Embodiment P-8. The method of any one of Embodiments P-1-P-7, wherein the level of PC4d is determined using an antibody specific for C4d, and the level of C3 is determined using an antibody specific for C3.

Embodiment P-9. The method of any one of Embodiment P-1-P-8, wherein the level of anti-PS/PT IgG antibodies are determined using an enzyme-linked immunosorbent assay (ELISA) in which ELISA plates are coated with PS/PT.

Embodiment P-10. The method of any one of Embodiments P-1-P-9, wherein the level of LAC is determined using dilute Russell's Viper Venom Time (dRVVT>37 seconds).

Embodiment P-11. The method of any one of Embodiments P-1-P-10, wherein the PC4d threshold level is ≥20 units measured using flow cytometry as described herein.

Embodiment P-12. The method of any one of Embodiments P-1-P-11, wherein the C3 threshold level is <81 mg/dl measured using an antibody specific for C3, as described herein.

Embodiment P-13. The method of any one of Embodiments P-1-P-12, wherein the anti-PS/PT IgG threshold level is >30 units measured using ELISA as described in Embodiment P-9.

Embodiment P-14. The method of any one of claims 1-13, wherein the subject is a human subject.

Embodiment P-15. The method of Embodiment P-14, wherein the human subject is female.

Embodiment P-16. The method of any one of Embodiments P-1-P-15, wherein the PC4d, C3, and one or both of the anti-PS/PT IgG antibody levels and the LCA levels are determined on 2, 3, 4, or more occasions.

Embodiment P-17. The method of any one of Embodiments P-1-P-15, wherein the PC4d, C3, and anti-PS/PT IgG antibody levels are determined on 2, 3, 4, or more occasions.

Embodiment P-18. The method of any one of Embodiments P-16-P-17, wherein a combination of (i) a level of PC4d in the biological sample above a threshold PC4d level, (ii) a level of complement C3 below a threshold C3 level, and (iii) a level of one or both of anti-PS/PT IgG antibodies above a threshold anti-PS/PT IgG antibody level and/or a level of LAC in the biological sample above a threshold LAC level on each of the 2, 3, 4, or more occasions indicates that the subject has risk thrombosis, is at risk of thrombosis, or indicate efficacy of the anti-thrombotic therapy.

Embodiment P-19. The method of any one of Embodiments P-1-P-18, wherein the subject is being treated with hydroxychloroquine (HCQ), and wherein the methods further comprise determining a level of HCQ in a whole blood sample from the subject, wherein an HCQ level below a threshold HCQ whole blood level indicates that the subject has venous thrombosis, is at risk of venous thrombosis, and/or indicates efficacy of HCQ and any other anti-thrombotic therapy.

Embodiment P-20. The method of Embodiment P-19, wherein the HCQ threshold is 500 ng/ml.

Embodiment P-21. The method of any one of Embodiments P-1-P-20, wherein the method further comprises communication of the diagnosis, prognosis, or indication of treatment effect via a remote patient monitoring (RPM) internet-based devices and/or application (including but not limited to a smartphone, smartwatch, or other device application) to a medical professional, including but not limited to the subject's doctor and/or the subject's pharmacy for automated ordering of an anti-thrombotic therapeutic, including but not limited to an oral anti-coagulant.

Embodiment P-22. The method of any one of Embodiments P-1-P-21, wherein the subject is identified as having thrombosis, at risk of thrombosis, or in need of modified therapy for thrombosis, wherein the method further comprises treating the subject with an anti-thrombotic therapeutic, or increasing the dosage of an anti-thrombotic therapeutic, including but not limited to hydroxychloroquine, heparin, dalteparin, fondaparinux, enoxaparin, warfarin, dabigatran, rivaroxaban, apixiban, or edoxaban.

### P-2 Embodiments

Embodiment P2-1. A method for diagnosing a risk of thrombosis in a subject having Systemic Lupus Erythematosus (SLE), comprising determining:

(a) a level of platelet-bound C4d (PC4d) protein in a biological sample from the subject;

(b) a level of complement C3 protein in a biological sample from the subject; and

(c) one or both of:

(i) a level of anti-phosphatidyl serine/prothrombin (PS/PT) IgG antibody in a biological sample from the subject; and/or

(ii) a level of lupus anticoagulant (LAC) in a biological sample from the subject;

wherein a combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the subject has a risk of thrombosis.

Embodiment P2-2. The method of Embodiment P2-1, comprising determining a level of anti-phosphatidyl serine/prothrombin (PS/PT) IgG antibody in a biological sample from the subj ect.

Embodiment P2-3. A method for prognosing development of thrombosis in a subject having Systemic Lupus Erythematosus (SLE), comprising determining a:

(a) a level of platelet-bound C4d (PC4d) protein in a biological sample from the subject;

(b) a level of complement C3 protein in a biological sample from the subject; and

(c) one or both of:

(i) a level of anti-phosphatidyl serine/prothrombin (PS/PT) IgG antibody in a biological sample from the subject; and/or

(ii) a level of lupus anticoagulant (LAC) in a biological sample from the subject;

wherein a combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the subject is at risk of developing thrombosis.

Embodiment P2-4. The method of Embodiment P2-3, comprising determining a level of anti-phosphatidyl serine/prothrombin (PS/PT) IgG antibody in a biological sample from the subject.

Embodiment P2-5. A method for monitoring treatment for thrombosis in a subject having Systemic Lupus Erythematosus (SLE) and being treated for thrombosis, comprising determining:

(a) a level of platelet-bound C4d (PC4d) protein in a biological sample from the subject;

(b) a level of complement C3 protein in a biological sample from the subject; and

(c) one or both of:

(i) a level of anti-phosphatidyl serine/prothrombin (PS/PT) IgG antibody in a biological sample from the subject; and/or

(ii) a level of lupus anticoagulant (LAC) in a biological sample from the subject;

wherein a combination of (i) a level of PC4d in the biological sample above a threshold PC4d level, (ii) a level of complement C3 below a threshold C3 level, and (iii) a level of one or both of anti-PS/PT IgG antibody above a threshold anti-PS/PT IgG antibody level and/or a level of LAC in the biological sample above a threshold LAC level, indicates that the treatment for thrombosis has not been effective; and/or

wherein a combination of (i) a level of PC4d in the biological sample at or below a threshold PC4d level, (ii) a level of complement C3 at or above a threshold C3 level, and (iii) a level of anti-PS/PT IgG antibody at or below a threshold anti-PS/PT IgG antibody level and/or a level of LAC in the biological sample above a threshold LAC level, indicates that the treatment for thrombosis has been effective.

Embodiment P2-6. The method of Embodiment P2-5, comprising determining:

(a) a level of platelet C4d (PC4d) marker in a biological sample from the subject;

(b) a level of complement C3 marker in a biological sample from the subject; and

(c) a level of anti-phosphatidyl serine/prothrombin (PS/PT) IgG antibody in a biological sample from the subject;

wherein a combination of (i) a level of PC4d marker in the biological sample above a threshold PC4d marker level, (ii) a level of complement C3 marker below a threshold C3 marker level, and (iii) a level of anti-PS/PT IgG antibody above a threshold anti-PS/PT IgG antibody level indicates that the treatment for thrombosis has not been effective; and/or

wherein a combination of (i) a level of PC4d marker in the biological sample at or below a threshold PC4d marker level, (ii) a level of complement C3 marker at or above a threshold C3 marker level, and (iii) a level of anti-PS/PT IgG antibody at or below a threshold anti-PS/PT IgG antibody level indicates that the treatment for thrombosis has been effective.

Embodiment P2-7. The method of any one of Embodiments P2-1 to P2-6, wherein the level of PC4d protein is in a whole blood biological sample.

Embodiment P2-8. The method of any one of Embodiments P2-1 to P2-7, wherein the level of complement C3 protein is in a serum sample.

Embodiment P2-9. The method of any one of Embodiments P2-1 to P2-8, wherein the level of anti-PS/PT antibody is in a serum, plasma, or whole blood sample.

Embodiment P2-10. The method of any one of Embodiments P2-1 to P2-9, wherein the level of PC4d is determined using an antibody specific for C4d, and the level of C3 is determined using an antibody specific for C3.

Embodiment P2-11. The method of any one of Embodiments P2-1 to P2-10, wherein the level of anti-PS/PT IgG antibody is determined using an enzyme-linked immunosorbent assay (ELISA) comprising ELISA plates, wherein the ELISA plates are coated with PS/PT.

Embodiment P2-12. The method of any one of Embodiments P2-1 to P2-11, wherein the level of LAC is determined to be positive.

Embodiment P2-13. The method of any one of Embodiments P2-1 to P2-12, wherein the threshold PC4d level is ≥20 mean fluorescence intensity (MFI) units measured using flow cytometry.

Embodiment P2-14. The method of any one of Embodiments P2-1 to P2-13, wherein the threshold C3 marker level is <81 mg protein per deciliter (mg/dl) serum measured using a C3-specific antibody.

Embodiment P2-15. The method of any one of Embodiment P2-1 to P2-14, wherein the threshold anti-PS/PT IgG level is >30 Units measured using ELISA.

Embodiment P2-16. The method of any one of Embodiments P2-1 to P2-15, wherein the subject is a human subject.

Embodiment P2-17. The method of any one of Embodiments P2-1 to P2-16, wherein the PC4d level, C3 level, and one or both of the anti-PS/PT IgG antibody and LAC levels are determined 2, 3, 4, or more times.

Embodiment P2-18. The method of any one of Embodiments P2-1 to P2-17, wherein the anti-PS/PT IgG antibody level is determined 2, 3, 4 or more times.

Embodiment P2-19. The method of any one of Embodiments P2-1 to P2-18, wherein the subject is being treated with hydroxychloroquine (HCQ), and wherein the method further comprises determining a level of HCQ in a whole blood sample from the subject, wherein an HCQ level below a threshold HCQ whole blood level indicates that the subject, is at risk of venous thrombosis, and/or indicates efficacy of HCQ and any other anti-thrombotic therapy.

Embodiment P2-20. The method of Embodiment P2-19, wherein the threshold HCQ whole blood level is 500 ng/ml.

Embodiment P2-21. The method of any one of Embodiments P2-1 to P2-20, wherein the method further comprises communication of the diagnosis, prognosis, or indication of treatment effect via a remote patient monitoring (RPM) internet-based devices to a medical professional, including but not limited to the subject's doctor and/or the subject's pharmacy for automated ordering of an anti-thrombotic therapeutic, including but not limited to an oral anti-coagulant.

Embodiment P2-22. The method of any one of Embodiments P2-1 to P2-21, wherein the subject is identified as having thrombosis, at risk of thrombosis, or in need of modified therapy for thrombosis, wherein the method further comprises treating the subject with an anti-thrombotic therapeutic, or increasing the dosage of an anti-thrombotic therapeutic selected from hydroxychloroquine, heparin, dalteparin, fondaparinux, enoxaparin, warfarin, dabigatran, rivaroxaban, apixiban, betrixaban and edoxaban.

Embodiment P2-23. A method of detecting a marker in a Systemic Lupus Erythematosus (SLE) subject that has or is suspected of having thrombosis, the method comprising determining:

(a) a level of platelet C4d (PC4d) protein in a biological sample from the subject;

(b) a level of complement C3 protein in a biological sample from the subject; and

(c) one or both of:

(i) a level of anti-phosphatidyl serine/prothrombin (PS/PT) IgG antibody in a biological sample from the subject; and/or

(ii) a level of lupus anticoagulant (LAC) in a biological sample from the subject.

Embodiment P2-24. The method of Embodiments P2-23, comprising determining a level of anti-phosphatidyl serine/prothrombin (PS/PT) IgG antibody in a biological sample from the subject.

Embodiment P2-25. The method of any one of Embodiments P2-23 to P2-24, wherein the level of PC4d protein is in a whole blood biological sample.

Embodiment P2-26. The method of any one of Embodiment P2-23 to P2-25, wherein the level of complement C3 protein is in a serum sample or plasma sample.

Embodiment P2-27. The method of any one of Embodiments P2-23 to P2-26, wherein the level of anti-PS/PT antibody is in a serum, plasma, or whole blood sample.

Embodiment P2-28. The method of any one of Embodiments P2-23 to P2-27, wherein the level of PC4d is determined using an antibody specific for C4d, and the level of C3 is determined using an antibody specific for C3.

Embodiment P2-29. The method of any one of Embodiments P2-23 to P2-28, wherein the level of anti-PS/PT IgG antibody is determined using an enzyme-linked immunosorbent assay (ELISA) in which ELISA plates are coated with PS/PT.

Embodiment P2-30. The method of any one of Embodiments P2-23 to P2-29, wherein the level of LAC antibody is determined to be positive.

Embodiment P2-31. The method of any one of Embodiments P2-23 to P2-30, wherein the threshold PC4d level is ≥20 mean fluorescence intensity (MFI) units measured using flow cytometry.

Embodiment P2-32. The method of any one of Embodiments P2-23 to P2-31, wherein the threshold C3 marker level is <81 mg protein per deciliter (mg/dl) serum or plasma measured using a C3-specific antibody.

Embodiment P2-33. The method of any one of Embodiments P2-23 to P2-32, wherein the threshold anti-PS/PT IgG level is >30 Units measured using ELISA.

Embodiment P2-34. The method of any one of Embodiments P2-23 to P2-33, wherein the subject is a human subject.

Embodiment P2-35. The method of any one of Embodiments P2-23 to P2-34, wherein the PC4d level, C3 level, and one or both of the anti-PS/PT IgG antibody and LAC levels are determined 2, 3, 4, or more times.

Embodiment P2-36. The method of any one of Embodiments P2-23 to P2-35, wherein the anti-PS/PT IgG antibody level is determined 2, 3, 4 or more times.

Embodiment P2-37. The method of any one of Embodiments P2-23 to P2-36, wherein the subject is being treated with hydroxychloroquine (HCQ), and wherein the method further comprises determining a level of HCQ in a whole blood sample from the subject, wherein an HCQ level below a threshold HCQ whole blood level indicates that the subject is at risk of venous thrombosis, and/or indicates efficacy of HCQ and any other anti-thrombotic therapy.

Embodiment P2-38. The method of Embodiment P2-37, wherein the threshold HCQ whole blood level is 500 ng/ml.

Embodiment P2-39. The method of any one of Embodiments P2-23 to P2-38, wherein the subject is identified as having thrombosis, at risk of thrombosis, or in need of modified therapy for thrombosis, wherein the method further comprises treating the subject with an anti-thrombotic therapeutic, or increasing the dosage of an anti-thrombotic therapeutic selected from hydroxychloroquine, heparin, dalteparin, fondaparinux, enoxaparin, warfarin, dabigatran, rivaroxaban, apixiban, betrixaban and edoxaban.

Embodiment P2-40. A method of treating thrombosis in a Systemic Lupus Erythematosus (SLE) subject comprising determining:

(a) a level of PC4d in a first blood sample from the subject; a

(b) a level of complement C3 protein in a second blood sample from the subject; and

(c) one or both of:

(i) a level of anti-phosphatidyl serine/prothrombin (PS/PT) IgG antibody in a third blood sample from the subject; and/or

(ii) a level of lupus anticoagulant (LAC) in a fourth blood sample from the subject, wherein the first, second, third and fourth blood samples may be the same or different; and

(d) treating the subject likely to have thrombosis with an effective amount of one or more antithrombotic therapeutic selected from hydroxychloroquine, heparin, dalteparin, fondaparinux, enoxaparin, warfarin, dabigatran, rivaroxaban, apixiban, betrixaban, and edoxaban.

Embodiment P2-41. The method of Embodiment P2-40, step (c) further comprising:

(iii) calculating a thrombosis risk score by adjusting the level of the markers by one or more transformation analyses, wherein the one or more transformation analyses comprises logistic regression analysis; and

(iv) comparing the thrombosis risk score to one or more of a standard thrombosis risk score.

Embodiment P2-42. A method for preparing a sample from a Systemic Lupus Erythematosus (SLE) subject useful for analyzing a plurality of markers involved in thrombosis, comprising:

(a) collecting whole blood from said subject;

(b) producing a platelet fraction derived from whole blood from said subject by comprising lysing red blood cells, and measuring a level of PC4d in said platelet fraction; and

(c) producing a serum or plasma fraction from the whole blood from said subject and measuring a level of C3 in said fractions; and

(d) producing a serum or plasma fraction from the whole blood from said subject and measuring a level of PS/PT complex antibody in said fraction.

Embodiment P2-43. The method of Embodiment P2-42, wherein said measuring the level of PC4d further comprises binding platelets using a platelet specific antibody.

Embodiment P2-44. The method of Embodiment P2-42 or Embodiment P2-43, wherein said measuring the level of PC4d further comprises fluorescence-activated cell sorting.

Embodiment P2-45. The method of one of Embodiments P2-42 to P2-44, wherein said measuring the level of C3 comprises an immunoturbidity assay.

Embodiment P2-46. The method of one of Embodiments P2-42 to P2-45, wherein said measuring the level of PS/PT complex antibodies comprises an immunoassay.

### EXAMPLES

### Example 1

The experiments conducted herein evaluated the relationships between PC4d and thrombosis in SLE, and built a composite score for thrombosis risk of PC4d, low complement C3 and lupus anticoagulant. Because of the difficulty in performing and interpreting the lupus anticoagulant assay in anticoagulated patients, anti-phosphatidyl serine/prothrombin [PS/PT] complex antibody was investigated as an alternative to LAC.

Patients

A cross-sectional study was designed to evaluate the association between complement abnormalities and a history of thrombosis in SLE, all enrolled at a single lupus center in the Baltimore area (Hopkins Lupus Center). All SLE fulfilled the 2012 Systemic Lupus International Collaborating Clinics (SLICC) Classification Criteria for SLE (see, for example, Ref. 10) and provided written informed consent to participate in the Johns Hopkins University School of Medicine IRB approved protocol that allowed the use of their samples and clinical data. Thrombosis that occurred in the past 5 years was classified as any thrombosis, venous thrombosis or arterial thrombosis, as described (see, for example, Ref 11). Disease activity was assessed using the Physician's Global Assessment (PGA) on a 0-3-point visual analogue scale and the Safety of Estrogens in Lupus Erythematosus National Assessment (SELENA) Systemic Lupus Erythematosus disease activity index (SLEDAI) without anti-dsDNA and complement components as described (Clinical SELENA-SLEDAI) (see, for example, Ref. 4). Active disease status was defined as a clinical SELENA-SLEDAI greater or equal to 4 points.

Methods

This was a cross sectional analysis of 149 consented SLE patients (mean age: 47±1 years, 86% females) classified with (n=16) or without (n=133) thrombotic events in the past 5 years. Abnormal PC4d (≥20 units) was measured using flow cytometry. LAC and C3 were measured using dilute Russell's Viper Venom Time (dRVVT>37 seconds) and immunoturbidimetry, respectively. Anti-PS/PT antibody status (IgG) was measured by immunoassay. Statistical analysis consisted of logistic regression and calculation of Odds ratio (OR) estimates with 95% confidence intervals (CI 95%).

Laboratory Markers

Whole blood and serum were collected in EDTA containing and serum separator tubes, respectively, and routed overnight to the reference clinical laboratory at Exagen (using transportation kit equipped with coolant cartridge). EC4d and BC4d levels were measured using fluorescence activated cell sorting (FACS) as described (see, for example, Ref 12), and expressed as net mean fluorescence intensity (MFI). PC4d levels were also measured by FACS as follow: red blood cells from EDTA whole blood were lysed and platelets were stained using mouse monoclonal antibody against human C4d (Quidel, San Diego, Calif.), or alternatively, using mouse IgG1 kappa monoclonal ([MOPC-21]), isotype control. After incubation for 30 minutes at 2-8° C., samples were stained using goat anti-mouse conjugated to FITC (30minutes at 2-8° C. in the dark). A monoclonal antibody against human CD42b conjugated to phycoerythrin (PE) (platelet specific marker) was used to identify C4d complement activation fragment covalently bound to the platelets. FACS analysis was performed using a Gallios (10-colors) flow cytometer (Beckman Coulter, Brea Calif.) equipped with an CXP software to measure fluorescent staining intensity. Light scatter (forward, and side) gating parameters were used during acquisition to isolate the platelet population followed by secondary gating based on positive CD42b PE staining. Quantification of the non-specific (isotype control) and specific (C4d) fluorescence in the FL1 (Fluorescein isothiocyanate) channel was determined for the CD42b PE gated platelet cells (5000 events). Net Mean fluorescence intensity (Net MFI) was determined by subtraction of isotype control background MFI results from the specific C4d MFI results on gated platelet cells. Abnormal EC4d, BC4d and PC4d status corresponded to levels greater than the 99^{th} percentile of normal healthy group (>14 and >60 and >20 net MFI, respectively) (see, for example, Ref. 3).

Antinuclear antibody (ANA) status was determined using digital Imaging on NOVA VIEW (≥1:80 as positive) (INOVA Diagnostics, San Diego, Calif.). Antibody titers to dsDNA were measured using chemiluminescence immunoassays (QUANTA Flash, INOVA Diagnostics). Low complement C3 or C4 status were established using serum C3 (<81.1 mg/dl) and C4 levels (<12.9 mg/dl) all measured using immunoturbidimetry (Optilite, The Binding Site, San Diego, Calif.). LAC was measured at the Hopkins Lupus Center using the dilute Russell's Viper Venom Time (dRVVT>37 seconds). Anti-cardiolipin, anti-beta2 Glycoprotein I antibody (IgM, IgG and IgA isotypes) and anti-phosphatidylserine/prothrombin complex antibodies (IgM and IgG) were measured using immunoassays (INOVA Diagnostics). Manufacturer cutoffs were used for all assays. Site investigator (MP) was blinded to all CB-CAPs throughout the study, and testing personnel was blinded to clinical status.

Statistical Analyses

The association of a history of thrombosis (venous, arterial, or any thrombosis) in the past 5 years with the laboratory measures was assessed using positive and negative likelihood ratio (LR) and Odds ratio (OR), all reported with 95% confidence interval (CI 95%). Univariate and multivariate logistic regression tests with thrombosis as the dependent variable and the biomarkers as independent predictors were used. The cumulative presence of abnormalities in composite scores was calculated and odds ratios (OR) for thrombosis (per unit change) was calculated. Differences between models were estimated using minimum Akaike information criterion (AIC). Mann-Whitney and Chi-square tests were employed as appropriate.

Results

The study enrolled 149 consecutive SLE (mean 48.3 years, 83% ANA positive [>1:80]). Sixteen of them (10.7%) had a history of thrombosis (venous or arterial) in the past 5 years. Venous thrombosis occurred in 10 patients (inclusive of 8 deep vein thrombosis/pulmonary embolism and 2 superficial thrombosis); arterial thrombosis occurred in 8 patients (inclusive of 3 myocardial infarctions, 2 cerebrovascular accidents, one digital gangrene and two other arterial events). Two patients experienced both venous and arterial events. Patient characteristics are presented in Table 1. In this cohort of 149 patients, BC4d status was unavailable in 9 patients due to low B cell events (<200) collected on the flow cytometer. Complement C3/C4 was not available in one patient. Among 140 patients with C3, C4, EC4d and BC4d status available, a 21.4% higher proportion of SLE were positive for abnormal EC4d or BC4d (42.9%, n=60/140) than for low complement C3 or C4 (21.4%, n=30/140) (p<0.01).

TABLE 1
Patient characteristics in the presence or
absence of any thrombosis (venous or arterial)
Results are expressed as percent and average (SEM) as appropriate.

| | | Absence of | Presence of |
|---|---|---|---|
| | All | thrombosis | thrombosis |
| Number of patients | 149 | 133 | 16 |
| Age | 48.3 ± 1.2 | 48.4 ± 1.3 | 47.5 ± 3.3 |
| Gender [% females] | 85.9% | 84.2% | 100.0% |
| Ethnicities | | | |
| Caucasians (%) | 55.7% | 59.4% | 25.0% |
| African Americans (%) | 34.2% | 32.3% | 50.0% |
| Asians (%) | 4.0% | 3.0% | 12.5% |
| Others (%) | 6.0% | 5.3% | 12.5% |
| PGA [0-3 cm] | 0.7 ± 0.1 | 0.6 ± 0.2 | 0.7 ± 0.2 |
| Clinical SELENA-SLEDAI | 1.3 ± 0.2 | 1.2 ± 0.2 | 1.8 ± 0.5 |
| Active disease status ≥4 | 18.2% | 16.7% | 31.2% |
| points (%) | | | |
| Treatment information | | | |
| Prednisone | 32.2% | 29.3% | 56.3% |
| hydroxychloroquine | 87.2% | 86.5% | 93.8% |
| azathioprine | 8.1% | 7.5% | 12.5% |
| mycophenolate | 22.8% | 24.0% | 12.5% |

A significantly lower frequency of thrombosis was observed in Caucasians (25% vs 59%; p=0.009) compared to other ethnic groups. African-Americans (32%; vs 50%; p=0.19) tended to have a higher frequency of thrombosis. Current prednisone treatment was also associated with a higher frequency of thrombosis (56% vs 32%). Patients taking prednisone were 3.1-fold more likely (OR=3.0 [C195%: 1.1-9.0]) (p=0.029) to have had thrombosis than those not taking prednisone. Other demographic variables, including active disease status (OR=2.2 [C195% 0.7-7.3]) (p=0.18), did not associate with thrombosis.

The frequency of laboratory abnormalities, overall, and by the presence of thrombosis (any thrombosis, venous thrombosis or arterial thrombosis) is highlighted in Table 2. Low C3, low C4, abnormal EC4d, PC4d, anti-PS/PT IgG and LAC status all significantly associated with any thrombosis (p<0.05).

TABLE 2
Laboratory measures in the presence or absence of any thrombosis (venous or arterial).
Likelihood ratio (LR) and Odds ratio (OR) are given with 95% confidence intervals (CI).

| | All | Absence of | Presence of | Positive | Negative | |
|---|---|---|---|---|---|---|
| | cohort | thrombosis | thrombosis | LR | LR | OR |
| | % (n/N) | % (n/N) | % (n/N) | (CI 95%) | (CI 95%) | (CI 95%) |
| Anti-dsDNA >35 U | 29.5% (44/149) | 27.1% (36/133) | 50.0% (8/16) | 1.85 (0.98-3.1) | 0.69 (0.38-1.01) | 2.69 (0.97-7.62) |
| Low C3 (<81 mg/dl) ^{a} | 11.5% (17/148) | 7.6% (10/132) | 43.7% (7/16) | 5.78 (2.49-12.38) | 0.61 (0.36-0.84) | 9.49 (3.01-30.26) ^{d} |
| Low C4 (<12.9 mg/dl) | 16.9% (25/148) | 14.4% (19/132) | 37.5% (6/16) | 2.61 (1.16-5.13) | 0.73 (0.45-0.97) | 3.57 (1.20-10.70) ^{d} |
| EC4d >14 net MFI | 33.6% (50/149) | 30.1% (40/133) | 62.5% (10/16) | 2.08 (1.22-3.10) | 0.54 (0.26-0.89) | 3.88 (1.36-11.03) ^{d} |
| BC4d >60 net MFI ^{b} | 27.7% (39/141) | 26.2% (33/126) | 40.0% (6/15) | 1.53 (0.72-2.74) | 0.81 (0.48-1.12) | 1.87 (0.64-5.51) |
| PC4d >20 net MFI | 21.5% (32/149) | 16.5% (22/133) | 62.5% (10/16) | 3.78 (2.08-6.22) | 0.45 (0.22-0.74) | 8.41 (2.84-24.78) ^{d} |
| LAC, dRVVT >37 s ^{c} | 59.7% (86/144) | 56.3% (72/128) | 87.5% (14/16) | 1.56 (1.11-1.91) | 0.29 (0.08-0.84) | 5.44 (1.31-22.34) ^{d} |
| Anti-cardiolipin IgM (>30 U) | 6.7% (10/149) | 6.8% (9/133) | 6.3% (1/16) | 0.92 (0.15-4.82) | 1.01 (0.76-1.10) | 0.92 (0.14-6.17) |
| Anti-cardiolipin IgG (>30 U) | 16.1% (24/149) | 15.8% (21/133) | 18.8% (3/16) | 1.19 (0.39-3.04) | 0.96 (0.67-1.14) | 1.23 (0.35-4.44) |
| Anti-cardiolipin IgA (>30 U) | 15.4% (23/149) | 13.5% (18/133) | 31.3% (5/16) | 2.31 (0.95-4.89) | 0.79 (0.51-1.01) | 2.90 (0.94-9.07) |
| Anti-beta 2 GP1 IgM (>30 U) | 2.7% (4/149) | 2.3% (3/133) | 6.3% (1/16) | 2.77 (0.40-17.6) | 0.96 (0.73-1.03) | 2.89 (0.39-22.11) |
| Anti-beta 2 GP1 IgG (>30 U) | 22.1% (33/149) | 21.1% (28/133) | 31.3% (5/16) | 1.48 (0.64-2.96) | 0.87 (0.56-1.11) | 1.70 (0.57-5.14) |
| Anti-beta 2 GP1 IgA (>30 U) | 8.1% (12/149) | 6.8% (9/133) | 18.8% (3/16) | 2.77 (0.84-8.08) | 0.87 (0.61-1.01) | 3.18 (0.93-12.50) |
| Anti-PS/PT IgM (>30 U) | 36.9% (55/149) | 34.6% (46/133) | 56.3% (9/16) | 1.63 (0.92-2.46) | 0.67 (0.35-1.05) | 2.43 (0.87-6.76) |
| Anti-PS/PT IgG (>30 U) | 21.5% (32/149) | 16.5% (22/133) | 62.5% (10/16) | 2.22 (1.16-3.70) | 0.64 (0.36-0.95) | 3.43 (1.22-9.67) ^{d} |

^{a} C3 was not available in one patient.
^{b} BC4d was not available in 8 patients due to low number of events;
^{c} LAC was not available in 4 patients;
^{d} p < 0.05

Median (inter-quartile range [IQR]) EC4d and PC4d levels were 2.2 and 5.5-fold higher, respectively, in the presence of thrombosis (20 net MFI [IQR: 9-59] and 27 net MFI [IQR:9-79], respectively) than in its absence (9 net MFI [IQR: 6-19] and 5 net MFI [IQR:2-14], respectively) (p<0.01). BC4d levels were slightly elevated in the presence of thrombosis (median 46 net MFI [IQR: 26-74] compared to 29 net MFI [IQR: 17-62]), but the difference was not significant (p=0.27).

Tables 3 and 4 highlight the associations between the laboratory measures and venous or arterial thrombosis. Low C3 and abnormal PC4d status associated with venous thrombosis (OR=10.5 [CI95%: 2.8-39.5] and 19.2 [CI95%: 4.2-84.7], respectively) (p<0.05) and arterial thrombosis (OR=5.4 [CI95%:1.28-23.17] and OR=4.0 [CI95%: 1.0-15.8], respectively) (p<0.05). All patients with venous thrombosis tested positive for LAC (OR=+∞[CI95%: 1.3-+∞], 100% sensitive) but LAC did not associate specifically with arterial thrombosis (OR=2.10 [CI95%: 0.5-9.4]; p=0.37). This contrasted with anti-PS/PT IgG status which associated with arterial (OR=5.4 [CI95%: 1.3-21.9]; p=0.02), but not venous thrombosis (OR=2.10 [CI95%: 0.6-7.3]; p=0.30).

TABLE 3
Laboratory measures and history of venous thrombosis.

| | No | Venous | Positive | Negative | Odds |
|---|---|---|---|---|---|
| | thrombosis | thrombosis | LR | LR | Ratio |
| | % (n/N) | % (n/N) | (CI 95%) | (CI 95%) | (CI 95%) |
| Anti-dsDNA >35 U | 27.3% (38/139) | 60.0% (6/10) | 2.19 (1.09 -2.48) | 0.55 (0.23-0.96) | 3.99 (1.13-13.97) ^{d} |
| Low C3 (<81 mg/dl)^{a} | 8.7% (12/138) | 50.0% (5/10) | 5.75 (2.35-11.99) | 0.55 (0.25-0.84) | 10.50 (2.81-39.53) ^{d} |
| Low C4 (<12.9 mg/dl) | 14.5% (20/138) | 50.0% (5/10) | 3.45 (1.50-6.49) | 0.58 (0.28-0.90) | 5.90 (1.65-21.07) ^{d} |
| EC4d >14 Net MFI | 30.9% (43/139) | 70.0% (7/10) | 2.26 (1.23-3.32) | 0.43 (0.15-0.89) | 5.21 (1.38-19.43) ^{d} |
| BC4d >60 net MFI ^{b} | 25.4% (34/132) | 55.6% (5/9) | 2.16 (0.99-3.61) | 0.60 (0.25-1.00) | 3.60 (0.98-13.22) |
| PC4d >20 net MFI | 17.3% (24/139) | 80.0% (8/10) | 4.63 (2.58-7.17) | 0.24 (0.07-0.62) | 19.17 (4.24-84.75) ^{d} |
| LAC, dRVVT >37 seconds^{c} | 57.7% (79/134) | 100.0% (10/10) | 1.76 (1.25-2.07) | 0.00 (0.00-0.65) | +∞ (1.93-+∞) ^{d} |
| Anti-cardiolipin IgM (≥20 U) | 6.5% (9/139) | 10% (1/10) | 1.54 (0.26-7.44) | 0.96 (0.64-1.07) | 1.60 (0.24-11.25) |
| Anti-cardiolipin IgG (≥20 U) | 15.8% (22/139) | 20% (2/10) | 1.26 (0.34-3.59) | 0.95 (0.58-1.15) | 1.33 (0.30-6.03) |
| Anti-cardiolipin IgA (≥20 U) | 14.4% (20/139) | 30% (3/10) | 2.08 (0.70-4.86) | 0.82 (0.46-1.06) | 2.55 (0.66-9.99) |
| Anti-beta 2 GP1 IgM (≥20 U) | 2.2% (3/139) | 10% (1/10) | 4.63 (0.67-1.01) | 0.92 (0.61-1.01) | 5.04 (0.66-40.57) |
| Anti-beta 2 GP1 IgG (≥20 U) | 22.3% (31/139) | 20% (2/10) | 0.90 (0.25-2.46) | 1.03 (0.63-1.26) | 0.87 (0.20-3.88) |
| Anti-beta 2 GP1 IgA (≥20 U) | 7.9% (11/139) | 10% (1/10) | 1.26 (0.21-5.92) | 0.98 (0.65-1.09) | 1.29 (0.20-8.89) |
| Anti-PS/PT IgM (>30 U) | 35.3% (49/139) | 60% (6/10) | 1.70 (0.86-2.61) | 0.62 (0.26-1.09) | 2.76 (0.78-9.58) |
| Anti-PS/PT IgG (>30 U) | 24.5% (34/139) | 40% (4/10) | 1.63 (0.66-3.12) | 0.79 (0.41-1.13) | 2.06 (0.59-7.28) |

^{a}C3 was not available in one patient.
^{b} BC4d was not available in 8 patients due to low number of events;
^{c}dRVVT was not available in 4 patients;
^{d} p < 0.05

TABLE 4
Laboratory measures and history of arterial thrombosis.

| | No | Arterial | Positive | Negative | Odds |
|---|---|---|---|---|---|
| | thrombosis | thrombosis | LR | LR | Ratio |
| | % (n/N) | % (n/N) | (CI 95%) | (CI 95%) | (CI 95%) |
| Anti-dsDNA >35 U | 28.4% (40/141) | 50.0% (4/8) | 1.76 (0.73-3.07) | 0.70 (0.30-1.12) | 2.53 (0.65-9.75) |
| Low C3 (<81 mg/dl)^{a} | 10.0% (14/140) | 37.5% (3/8) | 3.75 (1.25-8.75) | 0.69 (0.34-0.97) | 5.40 (1.28-23.17) ^{d} |
| Low C4 (<12.9 mg/dl) | 16.4% (23/140) | 25.0% (2/8) | 1.52 (0.42-4.03) | 0.90 (0.49-1.14) | 1.70 (0.37-7.97) |
| EC4d >14 Net MFI | 31.9% (45/141) | 62.5% (5/8) | 1.96 (0.93-3.04) | 0.55 (0.20-1.04) | 3.55 (0.89-14.13) |
| BC4d >60 net MFI ^{b} | 27.6% (37/134) | 28.6% (2/7) | 1.03 (0.29-2.50) | 0.99 (0.49-1.31) | 1.05 (0.22-4.97) |
| PC4d >20 net MFI | 19.9% (28/141) | 50.0% (4/8) | 2.52 (1.03-4.61) | 0.62 (0.27-0.99) | 4.04 (1.03-15.80) ^{d} |
| LAC, dRVVT >37 seconds^{c} | 58.8% (80/136) | 75.0% (6/8) | 1.27 (0.69-1.69) | 0.61 (0.17-1.49) | 2.10 (0.46-9.45) |
| Anti-cardiolipin IgM (≥20 U) | 6.4% (9/141) | 12.5% (1/8) | 1.95 (0.33-8.98) | 0.94 (0.56-1.06) | 2.10 (0.31-15.07) |
| Anti-cardiolipin IgG (≥20 U) | 15.6% (22/141) | 25.0% (2/8) | 1.60 (0.44-4.28) | 0.89 (0.48-1.12) | 1.80 (0.39-8.49) |
| Anti-cardiolipin IgA (≥20 U) | 14.2% (20/141) | 37.5% (2/8) | 2.64 (0.90-5.80) | 0.73 (0.35-1.02) | 3.63 (0.88-15.09) |
| Anti-beta 2 GP1 IgM (≥20 U) | 2.1% (3/141) | 12.5% (1/8) | 5.87 (0.86-34.27) | 0.89 (0.54-1.00) | 6.57 (0.85-54.87) |
| Anti-beta 2 GP1 IgG (≥20 U) | 20.6% (29/141) | 50.0% (4/8) | 2.43 (0.99-4.42) | 0.63 (0.27-1.00) | 3.86 (0.99-15.10) |
| Anti-beta 2 GP1 IgA (≥20 U) | 6.4% (9/141) | 37.5% (3/8) | 5.88 (1.85-15.07) | 0.67 (0.33-0.93) | 8.80 (2.00-39.94) ^{d} |
| Anti-PS/PT IgM (>30 U) | 36.2% (51/141) | 50.0% (4/8) | 1.38 (0.58-2.35) | 0.78 (0.33-1.26) | 1.76 (0.46-6.76) |
| Anti-PS/PT IgG (>30 U) | 23.4% (33/141) | 62.5% (5/8) | 2.67 (1.24-4.33) | 0.49 (0.18-0.92) | 5.45 (1.35-21.87) ^{d} |

^{a}C3 was not available in one patient.
^{b} BC4d was not available in 8 patients due to low number of events;
^{c}LAC was not available in 4 patients;
^{d} p < 0.05

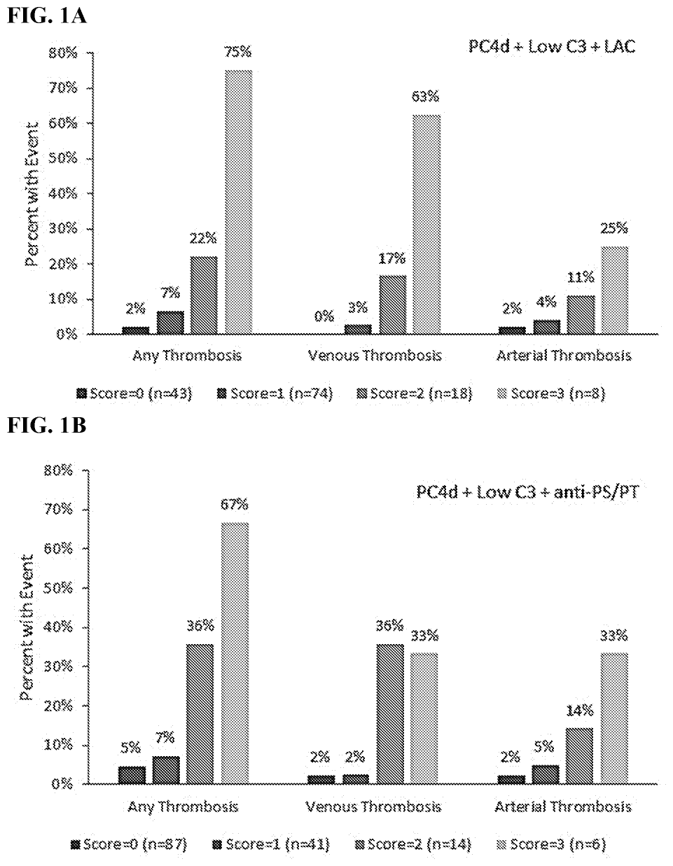

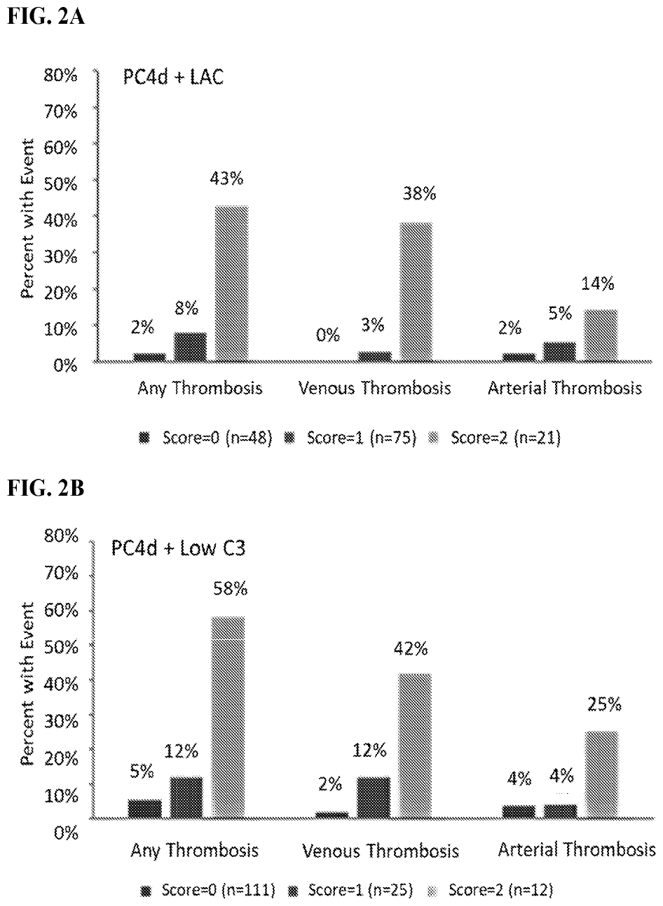

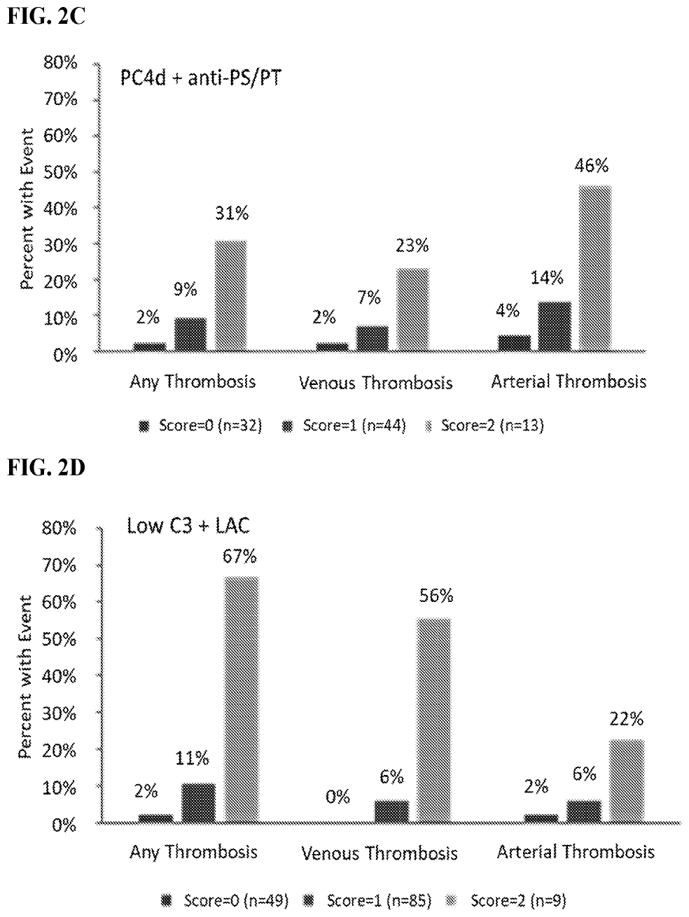

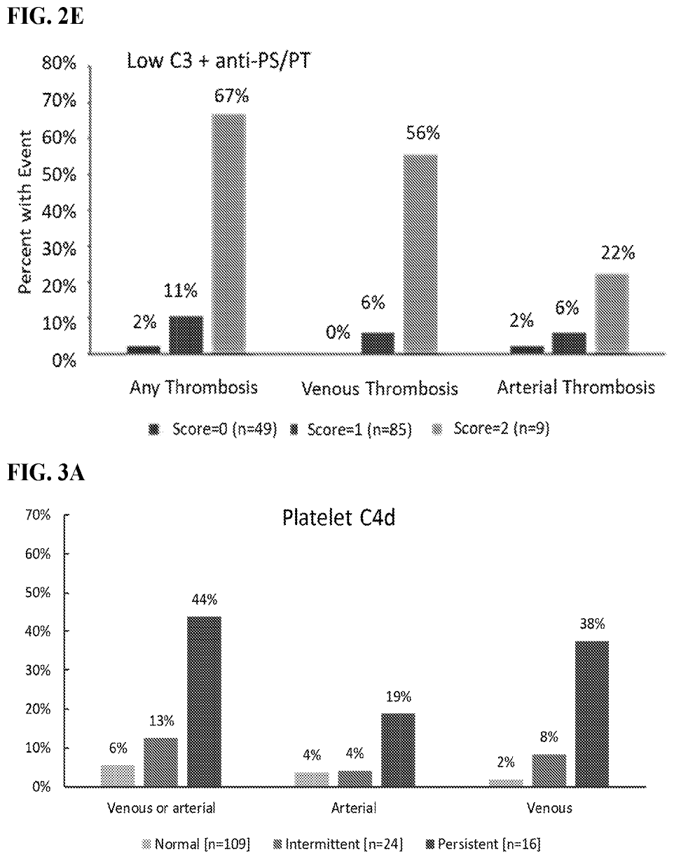

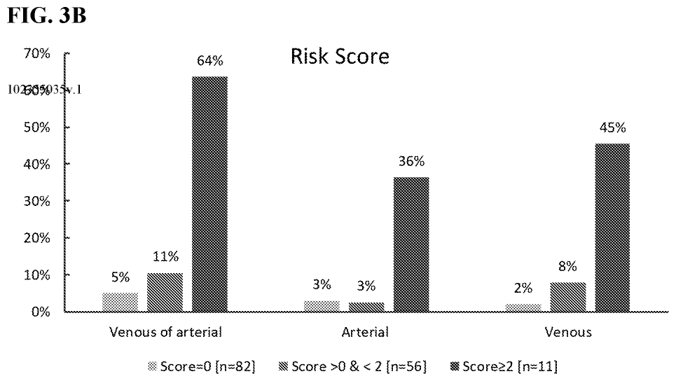

In 143 patients with PC4d, low C3 and LAC status available, multivariate logistic regression analysis revealed that abnormal PC4d (adjusted OR=4.2 [CI 95%: 1.1-16.0]; p=0.04), low C3 (adjusted OR=6.20 [CI95%: 1.3-29.6]; p=0.02) and LAC (adjusted OR=7.0 [CI95%: 1.3-38.5]; p=0.02) were all significantly and independently associated with any thrombosis, thus indicating additive utility of the three markers in combination. Cumulatively, the presence of PC4d, low C3 and LAC abnormalities as a composite risk score was higher in the presence of thrombosis (1.93±0.25) than in its absence (0.81±0.06) (p<0.01). Each unit of this composite risk score yielded an OR of 5.2 (CI95%: 2.5-10.7) to have thrombosis (p<0.01; AIC=78.94). (FIG. 1A). The average composite risk score was also higher in the presence of venous thrombosis (2.30±0.26 [n=10]) than in its absence (0.83±0.06 [n=133]) (p<0.001) and each unit of the score yielded an OR of 8.30 (CI95%: 3.16-21.83) for venous thrombosis (p<0.01; AIC=49.48). The composite risk score was also higher in the presence (1.62±0.37 [n=8]) of arterial thrombosis than in its absence (0.90±0.07 [n=135]) (p=0.05) and each unit of the score yielded an OR of 2.57 (CI95%: 1.17-5.64) for arterial thrombosis (AIC=60.27, p=0.02). Table 5 and FIG. 2A-E highlight the performances of other risk score combinations. PC4d, low C3 and LAC in combination yielded lower Akaike information criterion (AIC) for any thrombosis (78.94) and venous thrombosis (49.48) compared to other models.

TABLE 5
Markers and composite score in association with thrombosis.

| | Regression | | |
|---|---|---|---|
| | Estimate | OR | |
| | (SEM) | (CI 95%) | AIC |
| Venous or arterial events | | | |
| PC4d + low C3 | 1.53 (0.36) | 4.63 (2.28, 9.43) | 86.38 |
| PC4d + LAC | 1.96 (0.49) | 7.14 (2.7, 18.89) | 84.48 |
| PC4d + anti-PS/PT | 1.46 (0.39) | 4.29 (2.01-9.15) | 89.97 |
| Low C3 + anti-PS/PT | 1.54 (0.42) | 4.70 (2.05, 10.76) | 90.82 |
| Low C3 + LAC | 2.49 (0.64) | 12.05 (3.43, 42.35) | 83.19 |
| PC4d + low C3 + anti- | 1.28 (0.30) | 3.60 (1.98, 6.53) | 84.95 |
| PS/PT | | | |
| PC4d + low C3 + LAC | 1.64 (0.40) | 5.17 (2.5, 10.67) | 78.94 |
| Venous events | | | |
| PC4d + low C3 | 1.81 (0.45) | 6.14 (2.54, 14.84) | 58.69 |
| PC4d + LAC | 3.18 (0.81) | 23.97 (4.84, 118.74) | 50.46 |
| PC4d + anti-PS/PT | 1.49 (0.46) | 4.45 (1.79-11.04) | 66.08 |
| Low C3 + anti-PS/PT | 1.31 (0.48) | 3.72 (1.45, 9.53) | 69.76 |
| Low C3 + LAC | 3.11 (0.77) | 22.42 (4.99, 100.76) | 54.67 |
| PC4d + low C3 + anti- | 1.29 (0.35) | 3.65 (1.83, 7.27) | 62.15 |
| PS/PT | | | |
| PC4d + low C3 + LAC | 2.11 (0.49) | 8.30 (3.16, 21.83) | 49.48 |
| Arterial events | | | |
| PC4d + low C3 | 1.03 (0.44) | 2.82 (1.18, 6.75) | 61.24 |
| PC4d + LAC | 1.04 (0.57) | 2.84 (0.94, 8.61) | 62.18 |
| PC4d + anti-PS/PT | 1.30 (0.49) | 3.67 (1.39-9.67) | 59.20 |
| Low C3 + anti-PS/PT | 1.49 (0.53) | 4.47 (1.58, 12.67) | 58.21 |
| Low C3 + LAC | 1.34 (0.67) | 3.84 (1.03, 14.27) | 61.40 |
| PC4d + low C3 + anti- | 1.02 (0.36) | 2.79 (1.38, 5.65) | 58.18 |
| PS/PT | | | |
| PC4d + low C3 + LAC | 0.94 (0.40) | 2.57 (1.17, 5.64) | 60.27 |

OR with CI95% are provided with minimal AIC.
AIC: Akaike Information criteria.
PC4d: PC4d >20 net MFI; low C3: to C3 <81 mg/dl, LAC: dRVVT >37 seconds; anti-PS/PT: anti-PS/PT IgG >30 units.

Anti-PS/PT IgG as an alternative to LAC (n=148 patients) in composite risk score also yielded higher score in the presence versus the absence of any thrombosis (1.56±0.29 [n=16] vs 0.47±0.06 [n=132])(p<0.001), venous thrombosis (1.70±0.25 [n=10] vs 0.51±0.06 [n=138](p<0.001) and arterial thrombosis (1.50±0.25 [n=8] vs 0.53±0.06 [n=140](p=0.02). The odds ratio for any thrombosis was 3.60 (CI95: 1.98-6.53) (p<0.01; AIC=84.95), the odds ratio for venous thrombosis was 3.65 (1.83,7.27) (AIC=62.15), and the odds ratio for arterial thrombosis was 2.79 (CI95%: 1.38,5.65) (AIC=58.18) (FIG. 1B). PC4d, low C3 and anti-PS/PT in combination as composite risk score yielded a lower AIC (58.18) for arterial thrombosis than any of the other marker combinations (Table 5).

There was also a higher incidence of low C3 (26% vs 8%; p=0.001), low C4 (37% vs 12%; p=0.001), abnormal EC4d (59% vs 27%, p=0.001), PC4d (37% vs 18%), BC4d (59% vs 27%, p=0.08) and anti-dsDNA (48% vs 25%, p=0.01) among SLE presenting with active compared to inactive disease status. However, multivariate analysis with three CB-CAPs abnormalities revealed that only EC4d status (OR=3.5 [CI95%: 1.2-10.2]) (p=0.023) associated with active disease (BC4d: OR=1.2 [CI95%: 0.4-3.3] and PC4d: OR=1.3 [CI95%: 0.4-4.0]) (p>0.66). Antiphospholipid antibodies were generally not associated with active disease status (p>0.05; data not shown).

In summary, the results demonstrated that abnormal PC4d (OR=8.4 CI95%: 2.8-24.8), low C3 (OR=9.5 CI95%: 3.0-30.3), LAC (OR=5.4 CI95%: 1.3-22.3) and anti-PS/PT IgG (OR=3.4 CI95%: 1.2-9.7) status associated with thrombosis (p<0.05). Cumulatively, the presence of PC4d, low C3 and LAC abnormalities as a composite risk score was higher in the presence of thrombosis (1.93±0.25) than in its absence (0.81±0.06) (p<0.01). Each unit of this composite risk score yielded an OR of 5.2 (CI95%: 2.5-10.7) to have thrombosis (p<0.01). The composite risk score with anti-PS/PT antibody status instead of LAC also associated with thrombosis (p<0.01).

Discussion

SLE is an immune complex disease linked to classical complement pathway activation, hyper-consumption of C3 and C4 proteins and production of C4d split fragments covalently bound to a variety of hematopoietic cells including the erythrocytes, the B lymphocytes and the platelets. The complement system, platelets, and coagulation pathways interact. This is the first report of a composite risk index which includes measures of these pathways and which highlights the additive association with thrombosis in SLE.

PC4d yielded an OR of 8.4 for any thrombosis and the association of the marker for venous thrombosis was stronger (OR=19.2) than for arterial events (OR=4.0). In this study, 20% of SLE patients presented with abnormal C4d deposition on platelets (PC4d). Among cell bound complement activation products, PC4d is known to have the highest level of specificity for SLE. The data presented here 1) confirmed its value as a prognostic marker associating with thrombosis, and 2) added to the understanding that the complement system and platelets are intimately linked and co-operate in the development of venous and arterial thrombosis. In contrast, abnormal BC4d status was not associated with vascular events, and the weak contribution of EC4d status to thrombosis was negligible, after adjusting for the presence of PC4d.

LAC was a sensitive marker for thrombosis (75% for venous and 100% for arterial), yielding an odds ratio of 5.4 for any thrombosis. However, the statistically significant impact of LAC on thrombosis was restricted to venous events. This contrasted with anti-PS/PT antibody status (IgG isotype) that associated with arterial but not venous thrombosis.

In this cohort, low Complement C3 was the strongest risk factor for any thrombosis (OR=9.5) and associated with both venous (OR=10.5) and arterial thrombosis (OR=5.4). Clinical activity (physician global assessment or SLEDAI), in contrast, was not associated with thrombosis.

A multivariate analysis was conducted and combined the presence of low C3, PC4d and LAC to produce a composite score of risk factors for thrombosis. Independently, each of the three markers was significantly associated with thrombosis (adjusted OR ranging from 4.2 to 7.0), thus indicating additive value. We also estimated the value of anti-PS/PT as an alternative to LAC. While a lower odds ratio (3.6 vs 5.2 per unit change) was observed with anti-PS/PT, both composite scores with LAC or anti-PS/PT (as an aPL alternative) associated with venous and arterial thrombosis.

Prednisone was also associated with thrombosis, and these data are consistent with the elevated risk of thrombosis and increased damage in SLE. We have previously reported a dose-dependent association of prednisone with cardiovascular events in SLE (See, for example, Ref 1). While this is not completely understood, prednisone does increase Factor VIII levels, decreases fibrinolysis, and produces abnormal von Willebrand factor multimer composition (See for example, Ref. 2).

Collectively, these data as well as data from others strongly support the value of PC4d in associating with thrombosis. Both the composite risk score and the individual markers may be of value in clinical practice to identify patients likely to benefit from interventions, including hydroxychloroquine therapy, that can significantly reduce the risk of thrombosis in SLE (see, for example, Refs. 13-14).

In conclusion, a composite thrombosis risk equation including PC4d, low C3 and lupus anticoagulant strongly associated with thrombosis in SLE. This composite score of risk factors performed better than single risk factors alone.

### Example 2: Persistency in Platelet C4d and Thrombosis Risk Score Associate with Thrombosis in Systemic Lupus Erythematosus

A thrombosis risk score containing abnormal Platelet-bound C4d (PC4d), low complement C3 and abnormal anti-phosphatidyl serine prothrombin (PS/PT) IgG antibody was shown in Example 1 to associate with thrombosis in Systemic Lupus Erythematosus (SLE). The objective in Example 2 was to evaluate the relationships between persistency in PC4d, risk score and thrombosis during follow-up (FU). A secondary objective evaluated the impact of whole blood Hydroxychloroquine (HCQ) levels in associating with thrombosis.

Methods

This was a longitudinal study of 149 SLE patients (mean age: 47±1 years, 86% female), with (n=16, 11%) or without (n=132, 89%) a history of thrombosis (venous or arterial) in the past 5 years. PC4d was measured using flow cytometry. Percent FU visits with abnormal PC4d status (>20 mean fluorescence intensity [MFI]) was calculated. Persistency in PC4d was defined as abnormal PC4d (>20 net MFI) status at baseline and all FU visits, intermittent PC4d was defined as abnormal PC4d status during at least one visit. Complement C3 (<81 mg/dl) and anti-PS/PT IgG (>30 Units) were measured using immunoassays. Mean thrombosis risk score for each patient was calculated. Whole blood HCQ levels were measured using liquid chromatography and mean HCQ per patient was calculated. Statistical analysis consisted of Wilcoxon, Fisher's Exact and logistic regression. Odds Ratio (OR) with confidence intervals (CI) were calculated.

Results

424 follow up (FU) visits were collected (average 3 visits per patient). Persistent and intermittent PC4d status were observed in 16 (11%) and 24 patients (16%), respectively. Logistic regression analysis revealed that the percentage FU visits with abnormal PC4d status significantly associated with any thrombosis (OR range 11.7 CI 95%: 3.23-42.44) (p<0.001), venous thrombosis (OR range=33.4 CI95: 5.8-193.5) (p<0.001) and approached significance with arterial thrombosis (OR=4.7 CI 95%: 0.9-25.4) (p=0.08) (FIG. 1, panel A). During FU, the mean risk score per patient was 0.57±0.76 (n=149). Risk score at FU associated with any thrombosis (OR=3.8 CI 95%: 2.0-7.2 per unit change, OR range=53.9 CI 95%: 7.8-372.4) (p<0.001), venous thrombosis (OR=3.9 CI 95%: 1.9-8.2 per unit change, OR range=60.3 CI95%: 6.5-560.8) and arterial thrombosis (OR=3.0 CI95%: 1.4-6.4 per unit change, OR range=26.5 CI95%: 2.7-259.4) (FIG. 1, panel B). Among 133 patients treated with HCQ, median HCQ levels were 696 ng/ml (Interquartile range [IQR]: 537-989 ng/ml, n=15), 794 ng/ml (IQR: 628-1121 ng/ml, n=22), and 976 ng/ml (IQR: 675-1300 ng/ml, n=96) in the group of patients presenting with persistent, intermittent and normal PC4d status, respectively. Levels were significantly lower in the group of patients with persistent or intermittent PC4d status when compared to normal PC4d (p=0.028). Risk score did not associate with HCQ levels. Lower HCQ levels tended to associate with venous thrombosis (median=916 ng/ml [IQR: 675-1300], n=10 vs 648 ng/ml [IQR: 382-929], n=123) (p=0.061) but not with arterial thrombosis (p>0.20).

The data suggest that persistency in PC4d and the thrombosis risk score both associate with thrombosis. Lower HCQ levels may associate with venous thrombosis.

### Example 3: Summary and Anti-Thrombotic Therapeutics

As shown herein, the presence of PC4d, low C3, and one or both of anti-PS/PT and/LAC antibody status abnormalities as a composite risk score was significantly higher in the presence of thrombosis (1.93±0.25) than in its absence, and thus the methods provide a significant improvement in diagnosing, prognosing, and/or monitoring treatment for thrombosis in subjects having or at risk of SLE. The marker level combination is determined and used (or provided to a separate entity) to diagnose the subject as having thrombosis, prognose the subject as at risk of thrombosis, or indicate an efficacy of anti-thrombotic therapy.

In embodiments, as used herein, the “biological sample” is obtained from the subject's body. Any suitable biological sample from the subject may be used, and the biological sample from which each of the recited levels are determined may be the same or different. Particularly suitable samples for use in the methods of the invention are blood samples or serum samples. Blood samples are preferably treated with EDTA (ethylenediaminetetraacetate) to inhibit complement activation. Samples can be maintained at room temperature or stored at 4° C. In some embodiments, a whole blood sample may be fractionated into different components. For instance, in one embodiment, red blood cells are separated from other cell types in the sample by differential centrifugation. The platelet fraction can be from other blood components to allow analysis of platelet-bound complement activation products, such as PC4d. Platelet isolation can be performed with methods known in the art, including differential centrifugation or immunoprecipitation using antibodies specific for platelets (e.g., CD42b).

In embodiments, the level (e.g., quantity or amount) of a particular biomarker can be measured in the sample using a variety of methods known to those of skill in the art. Such methods include, but are not limited to, flow cytometry as described herein. In one embodiment, the determination of the level of PC4d and C3 is made using flow cytometric methods, with measurements taken by direct or indirect immunofluorescence using polyclonal or monoclonal antibodies specific for each of the molecules. Each of these molecules can be measured with a separate sample (e.g., platelet-specific fractions) or using a single sample (e.g., whole blood).

In embodiments, low complement C3 status may be established using any suitable method, including but not limited to those disclosed herein. In one embodiment, the biological sample comprises serum and C3 levels are measured using immunoturbidimetry. Anti-PS/PT complex antibodies may be measured using any suitable means, including but not limited to immunoassays.

In embodiments, the methods described herein employ comparisons between a measured level of PC4d, C3, and one or both of LAC and anti-PS/PT IgG antibody levels, and threshold levels of the same markers. Any suitable threshold for comparison can be used, including but not limited to a pre-determined threshold from an individual or population of normal or SLE subjects known to not have thrombosis. As used herein, a “pre-determined threshold” refers to a threshold value that can be determined from the quantity or amount (e.g., absolute value or concentration or mean fluorescence intensity) of a particular biomarker measured in a population of control subjects. A pre-determined threshold can be selected by calculating the value or range of values that achieves the greatest statistical significance for a given set of amounts or quantities for a particular biomarker.

In embodiments, “diagnosing/diagnosis,” as used herein, means identifying the presence or nature of thrombosis, while “prognosing” means predicting the development of thrombosis, and “monitoring” means following the course of thrombosis in response to treatment. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

In embodiments, a subject at risk of thrombosis is any subject that has one or more symptoms characteristic of thrombosis or has other characteristics that make the subject more likely to develop thrombosis. The thrombosis may be arterial or venous. Venous thrombosis leads to congestion of the affected part of the body, while arterial thrombosis affects the blood supply and leads to damage of the tissue supplied by that artery (ischemia and necrosis).

TABLE 6
Anti-thrombotic therapeutics

| | Dosing/route | | Mechanism of |
|---|---|---|---|
| Molecule | DVT prophylaxis | | action |
| Hydroxychloroquine | 100-600 mg/per day; | | |
| | often 400 mg/day | | |
| Heparin | 5000 | IU SC daily | Anti-thrombin III |
| Dalteparin (Fragmin) | 5000 | IU SC daily | low molecular weight |
| | | | heparin |
| Fondaparinux (Arixtra) | 2.5 | mg SC daily | low molecular weight |
| | | | heparin |
| Enoxaparin (Lovenox) | 4 | mg/day | low molecular weight |
| | | | heparin |
| Warfarin (Coumadin) | Individual INR 2-3 | | Anti-vitamin K |
| Dabigatran (Pradaxa) | 150 | mg orally | Direct thrombin |
| | | | inhibitor |
| Rivaroxaban (Xarelto) | 10 | mg daily | Direct Factor Xa |
| | | | inhibitor |
| Apixiban (Eliquis) | 2.5 | mg daily | Direct Factor Xa |
| | | | inhibitor |
| Edoxaban (Savaysa) | 60 | mg daily | Direct Factor Xa |
| | | | inhibitor |

### REFERENCES

1. Magder L S, Petri M. Incidence of and risk factors for adverse cardiovascular events among patients with systemic lupus erythematosus. Am.J.Epidemiol. 2012;176:708-719.

2. Girolami A, de Marinis G B, Bonamigo E, Treleani M, Vettore S. Arterial and venous thromboses in patients with idiopathic (immunological) thrombocytopenia: a possible contributing role of cortisone-induced hypercoagulable state. Clin.Appl.Thromb.Hemost. 2013;19:613-618.

3. Kalunian K C, Chatham W W, Massarotti E M et al. Measurements of cell bound complement activation products enhance diagnostic performance in systemic lupus erythematosus. Arthritis Rheum. 2012

4. Merrill J T, Petri M A, Buyon J et al. Erythrocyte-bound C4d in combination with complement and autoantibody status for the monitoring of SLE. Lupus Sci.Med. 2018;5:e000263.

5. Navratil J S, Manzi S, Kao A H et al. Platelet C4d is highly specific for systemic lupus erythematosus. Arthritis Rheum 2006;54:670-674.

6. Lood C, Tyden H, Gullstrand B et al. Platelet activation and anti-phospholipid antibodies collaborate in the activation of the complement system on platelets in systemic lupus erythematosus. PLoS.One. 2014;9:e99386.

7. Peerschke E I, Yin W, Alpert D R et al. Serum complement activation on heterologous platelets is associated with arterial thrombosis in patients with systemic lupus erythematosus and antiphospholipid antibodies. Lupus 2009;18:530-538.

8. Kao A H, McBurney C A, Sattar A et al. Relation of platelet C4d with all-cause mortality and ischemic stroke in patients with systemic lupus erythematosus. Transl.Stroke Res. 2014;5:510-518.

9. Akhter E, Shums Z, Norman G L et al. Utility of antiphosphatidylserine/prothrombin and

IgA antiphospholipid assays in systemic lupus erythematosus. J.Rheumatol 2013;40:282-286.

10. Petri M, Orbai A M, Alarcon G S et al. Derivation and validation of the Systemic Lupus International Collaborating Clinics classification criteria for systemic lupus erythematosus. Arthritis Rheum 2012;64:2677-2686.

11. Danowski A, de Azevedo M N, de Souza Papi J A, Petri M. Determinants of risk for venous and arterial thrombosis in primary antiphospholipid syndrome and in antiphospholipid syndrome with systemic lupus erythematosus. J.Rheumatol 2009;36:1195-1199.

12. Dervieux T, Conklin J, Ligayon J A et al. Validation of a multi-analyte panel with cell-bound complement activation products for systemic lupus erythematosus. J.Immunol.Methods 2017;446:54-59.

13. Fasano S, Margiotta D P, Navarini L et al. Primary prevention of cardiovascular disease in patients with systemic lupus erythematosus: case series and literature review. Lupus 2017;26:1463-1472.

14. Jung H, Bobba R, Su J et al. The protective effect of antimalarial drugs on thrombovascular events in systemic lupus erythematosus. Arthritis Rheum. 2010;62:863-868.

What is claimed is:

1. A method for diagnosing a risk of thrombosis in a subject having systemic lupus erythematosus (SLE), the method comprising determining:
(a) a level of platelet-bound C4d (PC4d) protein in a biological sample from the subject;
(b) a level of complement C3 protein in a biological sample from the subject; and
(c) one or both of:
(i) a level of anti-phosphatidyl serine/prothrombin (PS/PT) IgG antibody in a biological sample from the subject; and
(ii) a level of lupus anticoagulant (LAC) in a biological sample from the subject;
wherein a combination of (i) the level of PC4d protein in the biological sample above a threshold PC4d protein level, (ii) the level of complement C3 protein in the biological sample below a threshold C3 protein level, and (iii) one or both of the level of anti-PS/PT IgG antibody in the biological sample above a threshold anti-PS/PT IgG antibody level and the level of LAC in the biological sample above a threshold LAC level, indicates that the subject has a risk of thrombosis.

2. The method of claim 1, wherein (c) comprises determining the level of anti-phosphatidyl serine/prothrombin (PS/PT) IgG antibody in the biological sample from the subject.

3. A method for prognosing development of thrombosis in a subject having systemic lupus erythematosus (SLE), the method comprising determining:
(a) a level of platelet-bound C4d (PC4d) protein in a biological sample from the subject;
(b) a level of complement C3 protein in a biological sample from the subject; and
(c) one or both of:
(i) a level of anti-phosphatidyl serine/prothrombin (PS/PT) IgG antibody in a biological sample from the subject; and
(ii) a level of lupus anticoagulant (LAC) in a biological sample from the subject;
wherein a combination of (i) the level of PC4d protein in the biological sample above a threshold PC4d protein level, (ii) the level of complement C3 protein in the biological sample below a threshold C3 protein level, and (iii) one or both of the level of anti-PS/PT IgG antibody in the biological sample above a threshold anti-PS/PT IgG antibody level and the level of LAC in the biological sample above a threshold LAC level, indicates that the subject is at risk of developing thrombosis.

4. The method of claim 3, wherein (c) comprises determining the level of anti-phosphatidyl serine/prothrombin (PS/PT) IgG antibody in the biological sample from the subject.

5. A method for monitoring treatment for thrombosis in a subject having systemic lupus erythematosus (SLE) and being treated for thrombosis, comprising determining:
(a) a level of platelet-bound C4d (PC4d) protein in a biological sample from the subject;
(b) a level of complement C3 protein in a biological sample from the subject; and
(c) one or both of:
(i) a level of anti-phosphatidyl serine/prothrombin (PS/PT) IgG antibody in a biological sample from the subject; and
(ii) a level of lupus anticoagulant (LAC) in a biological sample from the subject;
wherein a combination of (i) the level of PC4d protein in the biological sample above a threshold PC4d protein level, (ii) the level of complement C3 protein in the biological sample below a threshold C3 protein level, and (iii) one or both of the level of anti-PS/PT IgG antibody in the biological sample above a threshold anti-PS/PT IgG antibody level and the level of LAC in the biological sample above a threshold LAC level, indicates that the treatment for thrombosis has not been effective; and/or
wherein a combination of (i) the level of PC4d protein in the biological sample at or below a threshold PC4d protein level, (ii) the level of complement C3 protein in the biological sample at or above a threshold C3 protein level, and (iii) one or both of the level of anti-PS/PT IgG antibody in the biological sample at or below a threshold anti-PS/PT IgG antibody level and the level of LAC in the biological sample above a threshold LAC level, indicates that the treatment for thrombosis has been effective.

6. The method of claim 5, comprising determining:
(a) the level of platelet C4d (PC4d) protein in the biological sample from the subject;
(b) the level of complement C3 protein in the biological sample from the subject; and
(c) the level of anti-phosphatidyl serine/prothrombin (PS/PT) IgG antibody in the biological sample from the subject;
wherein a combination of (i) the level of PC4d protein in the biological sample above the threshold PC4d protein level, (ii) the level of complement C3 marker in the biological sample below the threshold C3 protein level, and (iii) the level of anti-PS/PT IgG antibody in the biological sample above the threshold anti-PS/PT IgG antibody level indicates that the treatment for thrombosis has not been effective; and/or
wherein a combination of (i) the level of PC4d protein in the biological sample at or below the threshold PC4d protein level, (ii) the level of complement C3 protein in the biological sample at or above the threshold C3 protein level, and (iii) the level of anti-PS/PT IgG antibody in the biological sample at or below the threshold anti-PS/PT IgG antibody level indicates that the treatment for thrombosis has been effective.

7. The method of claim 1, wherein the level of PC4d protein is in a whole blood biological sample.

8. The method of claim 1, wherein the level of complement C3 protein is in a serum sample.

9. The method of claim 1, wherein the level of anti-PS/PT antibody is in a serum, plasma, or whole blood sample.

10. The method of claim 1, wherein the level of PC4d protein is determined using an antibody specific for C4d, and the level of complement C3 protein is determined using an antibody specific for C3.

11. The method of claim 1, wherein the level of anti-PS/PT IgG antibody is determined using an enzyme-linked immunosorbent assay (ELISA) comprising an ELISA plate, wherein the ELISA plate is coated with PS/PT.

12. The method of claim 1, wherein the level of LAC is determined to be positive.

13. The method of claim 1, wherein the threshold PC4d protein level is ≥20 mean fluorescence intensity (MFI) units measured using flow cytometry.

14. The method of claim 1, wherein the threshold complement C3 protein level is <81 mg protein per deciliter (mg/dl) serum measured using a complement C3-specific antibody.

15. The method of claim 1, wherein the threshold anti-PS/PT IgG antibody level is >30 Units measured using ELISA.

16. The method of claim 1, wherein the subject is a human subject.

17. The method of claim 1, wherein the PC4d protein level, complement C3 protein level, and one or both of the anti-PS/PT IgG antibody and LAC levels are determined 2, 3, 4, or more times.

18. The method of claim 1, wherein the anti-PS/PT IgG antibody level is determined 2, 3, 4 or more times.

19. The method of claim 1, wherein the subject is being treated with hydroxychloroquine (HCQ), and wherein the method further comprises determining a level of HCQ in a whole blood sample from the subject, wherein an HCQ level below a threshold HCQ whole blood level indicates that the subject is at risk of venous thrombosis and/or indicates efficacy of HCQ.

20. The method of claim 19, wherein the threshold HCQ whole blood level is 500 ng/ml.

21. The method of claim 1, wherein the method further comprises communication of the diagnosis, prognosis, or indication of treatment effect via a remote patient monitoring internet-based device to a medical professional and/or to a pharmacy for automated ordering of an anti-thrombotic therapeutic.

22. The method of claim 1, wherein the subject is identified as having thrombosis, at risk of thrombosis, or in need of modified therapy for thrombosis, wherein the method further comprises treating the subject with an anti-thrombotic therapeutic, or increasing the dosage of an anti-thrombotic therapeutic selected from hydroxychloroquine, heparin, dalteparin, fondaparinux, enoxaparin, warfarin, dabigatran, rivaroxaban, apixiban, betrixaban and edoxaban.

23. A method of detecting a marker in a Systemic Lupus Erythematosus (SLE) subject that has or is suspected of having thrombosis, the method comprising determining:
(a) a level of platelet C4d (PC4d) protein in a biological sample from the subject;
(b) a level of complement C3 protein in a biological sample from the subject; and
(c) one or both of:
(i) a level of anti-phosphatidyl serine/prothrombin (PS/PT) IgG antibody in a biological sample from the subject; and/or
(ii) a level of lupus anticoagulant (LAC) in a biological sample from the subject.

24. The method of claim 23, comprising determining a level of anti-phosphatidyl serine/prothrombin (PS/PT) IgG antibody in a biological sample from the subject.

25. The method of claim 23, wherein the level of PC4d protein is in a whole blood biological sample.

26. The method of claim 23, wherein the level of complement C3 protein is in a serum sample or plasma sample.

27. The method of claim 23, wherein the level of anti-PS/PT antibody is in a serum, plasma, or whole blood sample.

28. The method of claim 23, wherein the level of PC4d is determined using an antibody specific for C4d, and the level of complement C3 is determined using an antibody specific for complement C3.

29. The method of claim 23, wherein the level of anti-PS/PT IgG antibody is determined using an enzyme-linked immunosorbent assay (ELISA) in which ELISA plates are coated with PS/PT.

30. The method of claim 23, wherein the level of LAC antibody is determined to be positive.

31. The method of claim 23, wherein the threshold PC4d protein level is ≥20 mean fluorescence intensity (MFI) units measured using flow cytometry.

32. The method of claim 23, wherein the threshold complement C3 protein level is <81 mg protein per deciliter (mg/dl) serum or plasma measured using a C3-specific antibody.

33. The method of claim 23, wherein the threshold anti-PS/PT IgG antibody level is >30 Units measured using ELISA.

34. The method of claim 23, wherein the subject is a human subject.

35. The method of claim 23, wherein the PC4d protein level, complement C3 protein level, and one or both of the anti-PS/PT IgG antibody and LAC levels are determined 2, 3, 4, or more times.

36. The method of claim 23, wherein the anti-PS/PT IgG antibody level is determined 2, 3, 4 or more times.

37. The method of claim 23, wherein the subject is being treated with hydroxychloroquine (HCQ), and wherein the method further comprises determining a level of HCQ in a whole blood sample from the subject, wherein an HCQ level below a threshold HCQ whole blood level indicates that the subject is at risk of venous thrombosis, and/or indicates efficacy of HCQ and any other anti-thrombotic therapy.

38. The method of claim 37, wherein the threshold HCQ whole blood level is 500 ng/ml.

39. The method of claim 23, wherein the subject is identified as having thrombosis, at risk of thrombosis, or in need of modified therapy for thrombosis, wherein the method further comprises treating the subject with an anti-thrombotic therapeutic, or increasing the dosage of an anti-thrombotic therapeutic selected from the group consisting of hydroxychloroquine, heparin, dalteparin, fondaparinux, enoxaparin, warfarin, dabigatran, rivaroxaban, apixiban, betrixaban and edoxaban.

40. A method of treating thrombosis in a systemic lupus erythematosus (SLE) subject comprising determining:
(a) a level of platelet C4d (PC4d) protein in a first blood sample from the subject; a
(b) a level of complement C3 protein in a second blood sample from the subject; and
(c) one or both of:
(i) a level of anti-phosphatidyl serine/prothrombin (PS/PT) IgG antibody in a third blood sample from the subject; and/or
(ii) a level of lupus anticoagulant (LAC) in a fourth blood sample from the subject, wherein the first, second, third and fourth blood samples may be the same or different; and
(d) treating the subject likely to have thrombosis with an effective amount of one or more antithrombotic therapeutic selected from the group consisting of hydroxychloroquine, heparin, dalteparin, fondaparinux, enoxaparin, warfarin, dabigatran, rivaroxaban, apixiban, betrixaban, and edoxaban.

41. The method of claim 40, wherein step (c) further comprises:
(iii) calculating a thrombosis risk score by adjusting the level of anti-PS/PT IgG antibody and lupus anticoagulant by one or more transformation analyses, wherein the one or more transformation analyses comprises logistic regression analysis; and
(iv) comparing the thrombosis risk score to one or more of a standard thrombosis risk score.

42. A method for preparing a sample from a systemic lupus erythematosus (SLE) subject useful for analyzing a plurality of proteins involved in thrombosis, comprising:
(a) collecting whole blood from said subject;
(b) producing a platelet fraction derived from whole blood from said subject by comprising lysing red blood cells, and measuring a level of platelet C4d (PC4d) protein in said platelet fraction; and
(c) producing a serum or plasma fraction from the whole blood from said subject and measuring a level of complement C3 protein in said fractions; and
(d) producing a serum or plasma fraction from the whole blood from said subject and measuring a level of anti-phosphatidyl serine/prothrombin (PS/PT) IgG complex antibody in said fraction.

43. The method of claim 42, wherein said measuring the level of PC4d further comprises binding platelets using a platelet specific antibody.

44. The method of claim 42, wherein said measuring the level of PC4d further comprises fluorescence-activated cell sorting.

45. The method of claim 42, wherein said measuring the level of complement C3 protein comprises an immunoturbidity assay.

46. The method of claim 42, wherein said measuring the level of PS/PT IgG complex antibody comprises an immunoassay.
